(19) United States
(12) Patent Application Publication
(10) Pub. No.: US 2025/0239345 A1
(43) Pub. Date: Jul. 24, 2025

(54) SYSTEM AND METHOD TO PREDICT AND PRESCRIBE TREATMENTS FOR DISEASES

(71) Applicant: Incucomm, Inc., Addison, TX (US)

(72) Inventors: Toby Harrison Winter, Stamford (GB); Robert Ewan Michael Bennett, Lincoln (GB); Grace Elizabeth Walburn, Somerset (GB); Eric Haney, Dallas, TX (US)

(73) Assignee: Incucomm, Inc., Addison, TX (US)

(21) Appl. No.: 19/029,251

(22) Filed: Jan. 17, 2025

Related U.S. Application Data
(60) Provisional application No. 63/621,885, filed on Jan. 17, 2024.

(51) Int. Cl. G16H 20/10; G16H 10/60; G16H 20/30; G16H 50/20; G16H 50/80

(52) U.S. Cl. CPC G16H 20/10; G16H 10/60; G16H 20/30; G16H 50/20; G16H 50/80

(57) ABSTRACT

A system, and related method, operable on a processor and memory for predicting and prescribing a treatment to a disease for a patient, configured to collect population data about the disease, build a population and patient model based on the population data, record patient factors of the patient associated with the disease, and anonymize the patient factors by creating synthetic data of the patient factors. The system is also configured to merge the patient factors into the population and patient model, prescribe the treatment to the disease for the patient based on predicted outcomes from the population and patient model, and store the treatment to the disease for the patient in the memory.

### CROSS-REFERENCE TO RELATED APPLICATION

This application claims the benefit of U.S. Provisional Patent Application No. 63/621,885, entitled “System and Method to Quantify Underutilisation of Allogeneic Stem Cell Transplantation for Acute Myeloid Leukaemia,” filed Jan. 17, 2024, which is incorporated herein by reference.

This application is related to U.S. patent application Ser. No. 18/146,049 entitled “Adaptive Distributed Analytics System,” filed Dec. 23, 2022 (now U.S. Pat. No. 12,013,680 issued Jun. 18, 2024; U.S. patent application Ser. No. 18/050,661 entitled “System and Method for Adaptive Optimization,” filed Oct. 28, 2022; U.S. patent application Ser. No. 16/674,942 entitled “System and Method for Constructing a Mathematical Model of a System in an Artificial Intelligence Environment,” filed Nov. 5, 2019; U.S. patent application Ser. No. 16/947,555 entitled “Operations and Maintenance Systems and Methods Employing Sensor-less Digital Twins,” filed Aug. 6, 2020 (now U.S. Pat. No. 12,019,963 issued Jun. 25, 2024); U.S. patent application Ser. No. 18/475,963 entitled “System and Method for Real-Time Data Categorization,” filed Sep. 27, 2023; U.S. patent application Ser. No. 18/773,333 entitled “Generative Artificial Intelligence System and Method of Operating the Same,” filed Jul. 15, 2024; U.S. patent application Ser. No. 18/773,333 entitled “Continuous Asymmetric Risk Analysis System and Method of Operating the Same,” filed Jul. 22, 2024; and U.S. patent application Ser. No. 19/000,364 entitled “Synthetic Data Generation Systems and Applications,” filed Dec. 23, 2024, which are incorporated herein by reference.

### RELATED REFERENCES

Each of the cited references are incorporated herein by reference.
A Ten Year Vision for Stem Cell Transplantation and Cellular Therapies (2022), U.K. Stem Cell Strategic Forum.
Delivering on Data: Implementing our New Data Vision for HSCT and ATMP Data, (2023), U.K. Stem Cell Forum 2023 Data Workshop.
Loke, J. Alladi, R. Moss, P. Craddock, C. (Loke, et al., 2023) The role of allogeneic stem cell transplantation in the management of acute myeloid leukaemia: a triumph of hope and experience, British Journal of Haematology, https://pubmed.ncbi.nlm.nih.gov/31823351/.
Hanhua Liu, Simon J Stanworth, Sean McMphail, Mark Bishton, Brian Rous, Andrew Bacon, Thomas Coats (Hanhua, et al., Blood 2023) 1- and 5-Year Survival for Adults with Acute Myeloid Leukaemia and 30-Day Mortality after Initial Systemic Anti-Cancer Therapy, with an Exploration of Factors Associated with Poorer Outcomes: Data from a National Registry in England, 2013-2020.

### TECHNICAL FIELD

The present disclosure is directed, in general, to information systems and, more specifically, to remedial decision making systems, methods and applications to prescribe healthcare decisions to complex diseases.

### BACKGROUND

Acute Myeloid Leukemia (“AML”) is a highly aggressive type of blood cancer. Approximately 3000 cases are diagnosed annually just in the United Kingdom (“U.K.”) (over 20,000 new cases in the United States (“U.S.”)) with an average cure rate of just 15 percent (“%”) within five years of prognosis. Although this improves to a little over 50% in patients under the age of 40. The National Cancer Research Institute (“NCRI”) AML Working Party, made up of front-line clinicians and national experts, are looking for innovative technologies to improve patient outcomes.

Established in 2010, the U.K. Stem Cell Strategic Forum (“UKSCSF”) is tasked with producing recommendations that serve to improve outcomes for stem cell transplant patients. In 2022, the UKSCSF published its ten-year vision for stem cell transplantation and cellular therapies. A key part of this is to use data to drive improvements in patient outcomes.

For some patients, AML can be cured with chemotherapy alone. However, there is significant risk of relapse, and outcomes after disease relapse are very poor. The risk of disease relapse can be reduced (roughly 50%) through a curative transplant (or transplant) such as a bone marrow transplant (“BMT”) or an Allogeneic stem cell transplant (“Allo-SCT”) after chemotherapy, providing a suitable donor can be found. The curative transplant is a complex, highly specialized treatment that requires referral to a specialist center and is associated with significant toxicity and that can be fatal (35% non-relapse mortality), a debilitating treatment regime, and corresponding need for close and continual clinical monitoring. Therefore, the factors concerning a patient decision to undertake a curative transplant rely on not just survival, but also quality of life (“QoL”).

Patients experience different perspectives on how their own physiological, psychological, social, and economic circumstances will come into play as they move through their patient pathway. Being able to compare and understand these aspects is often constrained by limited access to information and limited access to a richly supporting decision framework.

While there is clinical trial data available, and several tools based on the probability of physiological success (survival) exist, the factors that impact survival and quality of life trade-offs have not before been captured and calculated. Nor has the wide variability, high uncertainty, and complex interplay of these factors been captured or calculated. This makes success (for the patient and the clinician) hard to define and predict, making the benefits and risks difficult to weigh up, explain and decide upon.

From this uncertainty, it is not clear whether some patients who could (and should) have further treatment are either not being referred or are being referred, but not electing to take up the treatment. It is also not clear if every patient, in an equal and equitable way, has been able to explore their pre-perceptions of treatments and received the personalized help they need to allow them to knowingly make the right decision for themselves and in their own terms. Medical professionals and patients need help to understand what factors make up a good decision based on the survival and quality of life for each patient.

There is also no standardized data-led decision framework for predicting the value of potential new interventions (whether new drugs or new practices) to improve patient outcomes, nor for assessing the most effective research methods to evaluate treatment interventions, nor for guiding best practice for introducing and using those treatments to best effect. The same data-led artificial intelligence (“AI”)-powered framework would also support stakeholders in optimizing the research and intervention investments for whole population-level patient-led outcomes through what-if analysis and predictions.

A curative transplant is a centrally important treatment in adults with AML in first complete remission (“CR1”). Despite advances in transplant technology and increased donor availability, concerns remain that this potentially curative modality is underutilized. It would be beneficial to employ an explainable AI model to estimate the under-referral of patients with AML in CR1 to gain an overall survival (“OS”) advantage from curative transplants.

### SUMMARY

Deficiencies of the prior art are generally solved or avoided, and technical advantages are generally achieved, by advantageous embodiments of the present disclosure of a system, and related method, operable on a processor and memory for predicting and prescribing a treatment to a disease for a patient, configured to collect population data about the disease, build a population and patient model based on the population data, record patient factors of the patient associated with the disease, and anonymize the patient factors by creating synthetic data of the patient factors. The system is also configured to merge the patient factors into the population and patient model, prescribe the treatment to the disease for the patient based on predicted outcomes from the population and patient model, and store the treatment to the disease for the patient in the memory.

The foregoing has outlined rather broadly the features and technical advantages of the present disclosure in order that the detailed description of the disclosure that follows may be better understood. Additional features and advantages of the disclosure will be described hereinafter, which form the subject of the claims of the disclosure. It should be appreciated by those skilled in the art that the conception and specific embodiment disclosed may be readily utilized as a basis for modifying or designing other structures or processes for carrying out the same purposes of the present disclosure. It should also be realized by those skilled in the art that such equivalent constructions do not depart from the spirit and scope of the disclosure as set forth in the appended claims.

### BRIEF DESCRIPTION OF THE DRAWINGS

For a more complete understanding of the present disclosure, reference is now made to the following detailed description taken in conjunction with the accompanying drawings, in which:

FIG. 1 illustrates a graphical representation of a proportion of patients transplanted compared to patients predicted to gain a survival benefit from a transplant in a first complete remission;

FIG. 2 illustrates a block diagram of an example patent journey;

FIG. 3 illustrates a diagram demonstrating the patient journey from diagnosis to living with and beyond cancer;

FIG. 4 illustrates a block diagram of a system to predict and prescribe treatments and remedies for diseases in the healthcare industry;

FIG. 5 illustrates block diagram of a system to predict and prescribe treatments and remedies for diseases in the healthcare industry;

FIGS. 6A to 6R illustrate exploded views of the sections of the system of FIG. 5;

FIG. 7 illustrates a block diagram of a system to predict and prescribe treatments and remedies for diseases in the healthcare industry; and

FIG. 8 illustrates a block diagram of an embodiment of an apparatus for operating systems and processes described herein.

Corresponding numerals and symbols in the different figures generally refer to corresponding parts unless otherwise indicated and, in the interest of brevity, may not be described after the first instance.

### DETAILED DESCRIPTION

After chemotherapy, eligible patients with Acute Myeloid Leukemia (“AML”) may not receive a curative transplant such as a bone marrow transplant (“BMT”) or an Allogeneic stem cell transplant (“Allo-SCT”) due to clinician or patient decisions. Various complex factors influence patient decision making and patient outcomes. There is a potential gap between the number of patients who could get treatment and the number of patients receiving it. Underpinning this issue is a lack of an artificial intelligence (“AI”) powered framework to support informed decision making. Additionally, there is no standardized framework for predicting the value of new interventions or optimizing research investments.

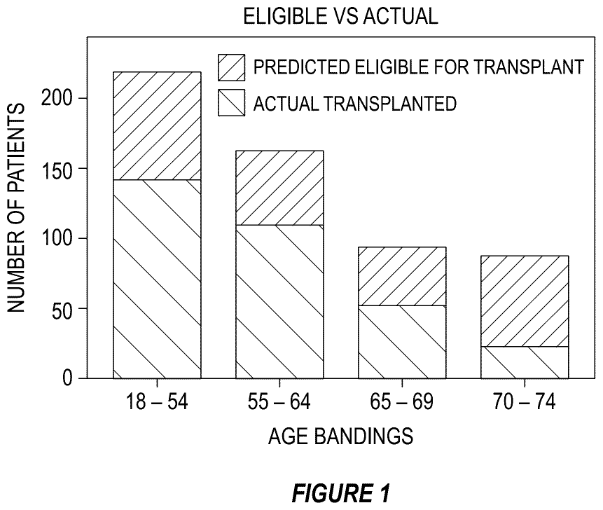

FIG. 1 illustrates a graphical representation of a proportion of patients transplanted compared to patients predicted to gain a survival benefit from a curative transplant in a first complete remission (“CR1”). The graphical representation plots the number of patients in the vertical axis within age bands on the horizontal axis. The system modelled a typical annual cohort of newly diagnosed patients. Five senior AML clinicians independently estimated relevant patient variables, including age, performance status, co-morbidities (hematopoietic cell transplantation-comorbidity index (“HCT-CI”)), disease biology and response rates. For each variable, the experts supplied five data points in percent, representing 0th, 10th, 50th, 90th, and 100th percentiles. Aggregated distributions were fed through a Bayesian inference network, representing each individual patient pathway. Running Monte-Carlo simulations, 50,000 cases were derived from the experts' answers to identify an expected range of patients in complete remission (“CR”).

The clinicians provided a threshold of HCT-CI score for recommending curative transplants (based on: European LeukemiaNet (“ELN”) risk group, minimal research disease (“MRD”) status, age (e.g., less than and greater than 40) and performance status (Eastern Cooperative Oncology Group (“ECOG”) (e.g., less than two, two and greater than two). Allo-SCT is rarely performed in patients greater than 75 years old so this demographic was excluded from transplant eligibility analysis. These thresholds were applied to the simulated patients to identify patients with potential overall survival (“OS”) benefit from CR1 Allo-SCT, this being compared to the number of AML CR1 allografts actually performed (data obtained from the British Society of Blood and Marrow Transplantation and Cellular Therapy (“BSBMTCT”), 2016-2020). Given that BSBMTCT data does not differentiate whether patients had intensive chemotherapy (“IC”) or non-intensive chemotherapy (“N-IC”) to achieve CR1, the experts provided what proportion of N-IC-treated patients treated achieving CR would be predicted to gain an OS benefit from Allo-SCT. Sensitivity analyses were performed to determine the impact of uncertainty of each variable on the model output.

The sensitivity analyses identified highest variability in clinicians' estimates of the number of AML cases/year and the age distribution at diagnosis. To improve accuracy of the model, clinician estimates were replaced with published incidence data (Cancer U.K.) and age distribution from National Cancer Registry and Analysis Service data (Hanhua, et. al, Blood 2023). All other variables had less than or equal to 10% variation above or below the model prediction.

The updated model predicts a median of 569 U.K. adult patients/year should derive improved OS from Allo-SCT in CR1 (95% confidence interval (“CI”) range 477-669), 443 following induction with IC and 126 following N-IC. Of course, the results may vary per run of the model. This is significantly higher than the U.K. Allo-SCT rate of 318/year. As demonstrated in FIG. 1, the proportion of patients predicted to be transplant-eligible who did not have Allo-SCT in CR1 was highest in the older age groups; 36% aged 18-54, 33% aged 55-64, 44% aged 65-69, 75% aged 70-74.

TABLE 1 illustrates an example transport eligibility look up table (Loke, et al., 2023).

TABLE 1

| | | | | Maximum | |
|---|---|---|---|---|---|
| | | | | tolerated | |
| | | | | non-relapse | Maximum |
| | | | | mortality | tolerated |
| | | | | (“NRM”) | NRM |
| | | | | prognosis | prognosis |
| | | Estimated | Estimated | scores for | scores for |
| 2017 ELN | | risk of relapse | risk of | Allo-SCT to | Allo-SCT to |
| risk | MRD after | with | relapse with | be beneficial | be beneficial |
| stratifications | cycle 2 | chemotherapy | Allo-SCT | HCT-C1 | NRM score |
| by genetics | chemotherapy | alone (%) | (%) | score | (%) |
| Favorable | Negative | 25-35 | 15-20 | N/A (less | 5 |
| | | | | than 1) | |
| | Positive | 70-80 | 30-40 | Less than or | Less than 30 |
| | | | | equal to 3-4 | |
| Intermediate | Negative | 50-60 | 25-30 | Less than or | Less than 20 |
| | | | | equal to 2 | |
| | Positive | 70-80 | 30-40 | Less than or | Less than 30 |
| | | | | equal to 3-4 | |
| Adverse | N/A | Greater than | 45-55 | Less than 5 | Less than 35 |
| | | 90 | | | |

The AI model suggests that the annual transplant rate in the U.K. should be almost 80% higher than observed. There is substantial inter-expert agreement on the size of the transplant eligible population to support these findings. These data provide the basis to examine why there is ongoing under-transplantation.

A framework, with a supporting tool, that addresses the complexities and uncertainties in a coherent and understandable way is the subject of the system herein. A ‘Patient-Focused Quality of Life Decision Support Tool’ (or tool or system) allows both patients and clinicians to navigate these difficult decisions and conversations. By explaining and exploring with the patient relationship between the potential improved survival benefit, the risks to survival of each option, the impacts on quality of life when receiving (or not receiving) a curative transplant, and what factors influence both the outcomes and their perceptions and experiences, it will help ensure that outcomes are in the patients' best overall interests.

The NCRI AML Working Party are pursuing the development of a patient registry. This will create a record of patient journeys that reflect their experiences before, during and after treatments. However, what to capture, how to capture it, and what best to use the data for once it has been captured, are still in active discussion. A Patient Focused Quality of Life Decision Support Tool may also contribute to a virtuous circle in the NCRI AML Working Party. Not only could this tool represent an ideal use of the data captured for the patient registry, but it could also play an active part in driving what data within the patient registry is best to gather.

In January 2023, the UKSCSF held a workshop called ‘Delivering on data: Implementing our new vision to hematopoietic stem cell transplantation (“HSCT”) and advanced therapy medical products (“ATMP”) data’ (summarized in TABLE 2). The vision identifies how the hematopoietic stem cell transplantation-computed tomography (“HSCT-CT”) community can enable the real-time identification of improvements and adaptations to clinical practice that support improved patient outcomes, experiences, and quality of life. The application of machine learning can transform clinical decision making by helping to ensure treatment decisions are personalized and provide the optimal chance of survival and return to a good quality of life. The U.K. HSCT-CT data can become a secure national asset that attracts investment into HSCT and ATMP research and innovation from the global life sciences sector.

TABLE 2
A new vision for HSCT and ATMP data

The BSBMTCT should make up-to-date outcomes data for each
HSCT-CT center publicly available on an annual basis, to
achieve greater transparency for patients and empower them
to make informed decisions about their care.
The Department of Health and Social Care and National
Health Service (“NHS”) England, and their
devolved counterparts, should ascertain the resource
necessary to collect high-quality HSCT and ATMP patient
data, and a mechanism should be established that ensures
HSCT-CT centers receive allocated funding to enhance
their data management capacity.
A new digital quality of life data tool should be developed
by the HSCT-CT community to collect self-reported experience
and quality of life data for HSCT and ATMP patients.
An HSCT-CT Data Change Commission should be created, as a
collaboration between the BSBMTCT, U.K. aligned registry
partners, NHS England and devolved counterparts, patients,
academia and industry, to create an enhanced, accessible and
sustainable data model for the U.K. HSCT-CT data.
The U.K. HSCT-CT data community should explore ethical
approaches to licensing datasets, to develop a sustainable
investment model for data infrastructure and further strengthen
the national HSCT-CT dataset into an asset that supports
improving patient outcomes and development of new therapies.
A new outcome-predictive clinical decision making tool
employing machine learning technologies to HSCT-CT data to
drive greater personalization of care to empower patients
receiving HSCT and ATMP therapies.

The Forum agreed on three broad strategic objectives that outlined a data model capable of providing a full picture of what they do, how they do it, and who they support. One objective is for greater accessibility that can support better clinical decision making with increased predictability, risk reduction for supply chains and clinical pathways, and enhanced patient experiences (summarized in TABLE 3).

TABLE 3
ACCESSIBILITY

| Activity | Enable the Activity |
|---|---|
| Make data publicly available to | Present data in a useable |
| help patients and their families | framework at the point of |
| understand HSCT and ATMPs, and | need in ways that make sense |
| make informed decisions. | for patients. |
| Give clinical teams the evidence | Provide a framework for |
| they need to improve the quality | evaluating experiences and |
| of care. | outcomes across all factors for |
| | individual patient-led assessments. |
| Grant clinical and academic | Provide a framework for |
| researches secure and defined | cross-population evaluation of |
| access to multiple datasets, | interventions and potential |
| supporting cross-reference queries | impact on new practices or |
| to develop translatable | medicines. |
| research findings. | |
| Engage patients in their own | The ability to feed the data |
| healthcare monitoring and | back into the framework and tool |
| management, using sensors such | to further personalize explanations |
| as wearables or mobile devices | and predictions as the patient's |
| to record observations, complete | pathway progresses. |
| longitudinal surveys or | |
| free-text logs. | |
| Permit industry secure commercial | Enable a shared language and |
| access using trusted platforms | model of physiological, |
| with comprehensive ethical | psychological, social and |
| approval and oversight. | economic factors that impact |
| | survivability and QoL. |

Another objective is for further integration of existing or prospective datasets based upon a federated ownership model, maintaining current data controllers and taking advantage of more data linkage with specific data processing permissions across the model (summarized in TABLE 4).

TABLE 4
INTEGRATION

| Activity | Enable the Activity |
|---|---|
| Developing a comprehensive view of | Provide a per-patient and |
| the full HSCT or ATMP patient pathway, | per-population predictive |
| from diagnosis to recovery, supporting | model to determine likely |
| analysis on health inequalities, | impacts of existing or new |
| unmet needs, and non-HSCT-CT centre | constraints and interventions. |
| interventions in the patient's | |
| recovery pathway. | |
| Full end-to-end data chain visibility, | A predictive framework to |
| enabling full monitoring and service | determine where changes are |
| improvement of multiple supply chains, | likely to have the biggest |
| including unrelated donor grafts and | impact on positive patient |
| personalized medicines, with cross-sector | outcomes, leading to |
| oversight to manage changes in lead-in | supporting prioritized |
| times and patient experience. | supply chain monitoring. |
| Digital compatibility with the latest | An open data approach, both |
| industry standards and emerging health | following and guiding data |
| data platforms across all U.K. nations, | use and data generation best |
| and internationally with respect to | practices. |
| transplant specific data assets and | |
| resources. | |

Another objective is sustainability for building a firm foundation for the long-term investment and development of HSCT-CT data management and analytics in the U.K. and being able to realize the potential for a data model to become a significant national life sciences asset (summarized in TABLE 5).

TABLE 5
SUSTAINABILITY

| Activity | Enable the Activity |
|---|---|
| Enable discovery research and development | Generating scenarios based |
| of new therapeutics (application of remedies | to support evaluation and |
| to diseases), by both private and public | business case development. |
| innovators, supporting sustainable pipelines | |
| and generating income for capital | |
| investment. | |
| Help NHS commissioners identify clinical | Support scenario evaluation |
| service investment opportunities, and to | and business case |
| provide appropriate funding for sustainable | development. |
| data management that supports clinical | |
| outcomes analysis and research studies. | |
| Use the new data model to establish co- | Integrate trial data into |
| ownership of pharmaceutical trial data, | patient pathway feasibility |
| ethically safeguarding the value of | and develop application |
| patients' data and re-invest in | guidance based on |
| future clinical research opportunities | simulations and |
| across the U.K. so every HSCT-CT patient | patient-led feedback. |
| can be offered a clinical trial or | |
| research study. | |

The Patient-Focused Quality of Life Decision Support Tool supports these objectives. The Forum also identified a set of timeframes in which to achieve the strategy. It identifies the development of a decision making model and a patient quality of life tool as a medium-to-long-term goal. The proposal study suggests this tool could be realized much more quickly. The framework optimizes patient pathway decisions and new intervention options into the patient pathways. There are patients that could receive better outcomes if there was a framework that accounted for the trade-offs between the quality of life and mortality, in terms of the physiological, psychological, social, and economic factors at play. This would aid the conversation and decision making process between clinicians, care providers and patients.

The Patient-Focused Quality of Life Decision Support Tool, tool or system identifies AML population statistics and maps the whole process from diagnosis, to chemotherapy, to patient transplant eligibility. The system identifies and engages patients before a curative transplant to provide insight on the patient factors affecting decisions for transplant uptake. The system collects data on patient-reported expected outcomes before elective treatment. The system identifies and engages AML practitioners to provide insight on the factors affecting decisions for referral. The system develops a comprehensive data driven model that captures patient-reported expected outcomes for elective treatment, and collects data on patient-reported actual outcomes following elective treatment. The system compares and analyzes the expected versus actual outcomes to determine any disparities, and predicts the degree to which the factors impact treatment decisions. The system proposes strategies such as providing a patient toolkit to empower patients by improving patient understanding and decision making regarding elective treatment.

The data collection and analysis includes interviews with doctors, nurses, hematologists, transplant doctors to design questions for patients and patient advocacy groups. Once the questions are answered, data is analyzed and missing data is captured to understand the factors for the missing data and work the questions into thoughts for the registry initial work suggests that there is little data available on comorbidities, impact of sex on outcome, which patients did or didn't have chemotherapy (also referred to as “chemo”). The model building and improvement includes inputting the collected data into the model, consolidating the model, and evaluating the model.

Example questions for a particular example are set forth below. Unless otherwise noted, the following questions typically request lower bound (the number will never be lower than this), 10th percentile (1 in 10 times a number lower than this), median (50% of the time to expect the number to be higher, 50% of the time lower), 90th percentile (1 in 10 times a number higher than this) and upper bound (the number will not be higher than this).
1. What is the age of patients diagnosed with AML?
2. What is the proportion of patients that have chemotherapy treatment (for selected age groupings)?
3. For newly diagnosed AML, what is the ratio of patients receiving intensive: non-intensive treatment for selected age groupings? NB Non-intensive treatment refers to Ven based, aza or low dose (“LD”) cytarabine based treatments.
4. For newly diagnosed AML, what is the distribution of ELN Risk Groups for selected age groupings (favorable, intermediate, adverse)?
5. What is the breakdown of molecular subgroups within favorable risk patients (as a percentage of all favorable risk patients—groups CBF, CEBPA, NPM1)?
6. What is the breakdown of molecular subgroups within intermediate risk patients (as a percentage of all intermediate risk patients—group NPM1)?
7. What is the breakdown of molecular subgroups within adverse risk patients (as a percentage of all adverse risk patients—group NPM1)?
8. What proportion (in percentage) of patients with NPM1 positive disease have a high white blood count (“WBC”) at diagnosis? NB the threshold you use to define a high WBC is arbitrary and will depend on the level you use in your own clinical practice to identify patients that may be at higher risk of relapse in NPM1 disease in complete remission.
8. What proportion of NPM1 patients are DNA Methyltransferase 3 Alpha (“DNMT3A”) positive at diagnosis?
10. What is the spread of performance status scores (ECOG scores 0-1, 2, 3-4) by selected age group at diagnosis of those having intensive chemotherapy?
11. What is the likelihood of dying in percentage during intensive chemotherapy by age/performance status (“PS”) grouping? NB this is referring to mortality during the initial phases of chemo undertaken to secure the ‘best’ possible response/the time point at which a decision to proceed to transplant is made. In reality for majority of patients this will be on completion of the first 2 cycles of chemotherapy. The model has not included adverse karyotype as a predictor of early mortality, to save having too many rows to fill out, so bear in mind that there will be a greater proportion of adverse karyotypes in your older age groups that may influence your assessment of mortality risk.
12. What is the likelihood in percentage of getting into complete remission from intensive chemotherapy by ELN Risk Group (excluding those patients who died during intensive chemo)—favorable, intermediate and adverse for age groupings?
13. For those alive and in complete remission after induction chemotherapy what is the likelihood of achieving MRD Negativity in percentage for each patient subgroup? The modality of MRD assessment and time point should be those usually used in clinical practice, e.g., PB molecular MRD after 2cycles for NPM1 mutation, flow MRD after 2 cycles for patients with no molecular target, possibly cycle 2 or more for CBF AML.
14. For those patients who are alive in CR1, what is the probability that their performance status changed significantly during intensive chemotherapy? For instance, some patients will have a deterioration of performance status from time of diagnosis to the time of achieving remission. Others will have an improvement (i.e., if very sick when first presented).

In several age groups the study looks at:
1. of those with a good performance status (ECOG 0-1) at diagnosis and who reach a complete remission, what is proportion in percentage who then have a poorer performance status (ECOG 2+) at that time point?
2. of those with a poorer performance status (ECOG 2+) at diagnosis and who reach a complete remission, what is proportion in percentage who then have an improved PS (ECOG 0-1) at that time point?
15. What is the spread of HCT-CI Score by age group/PS of those patients alive in CR1? Please write the percentage of patients who have each HCT-CI score for each grouping, i.e., each row should add up to 100%.
16. Of all patients transplanted for AML in the U.K., what is the proportion in percentage of those who are transplanted in CR1 (as opposed to CR2+, or with active disease)?
17. Total Number of Diagnosed Patients in a year in U.K. (using the more traditional cut off of 20% blasts rather than the International Consensus Classification (“ICC”) of myeloid neoplasms and acute leukemia has updated the classification myelodysplastic syndromes (“MDSs”)/AML 10% threshold)?
18. What is the proportion of patients who will survive to achieve a complete remission from Non-Intensive Chemotherapy?
19. Of those patients treated non-intensively who are alive in CR1, what proportion of these would be considered suitable for a transplant?

Next set of questions looks to establish a HCT-CI threshold for identifying patients where a survival benefit could be reasonably expected for a patient in CR1 (based on disease, treatment response and patient factors). This is to identify the cohort of patients who could be referred for consideration of a curative transplant (assuming a suitable donor could be found, the patient was willing to undergo transplant, etc.) Please provide the maximum HCT-CI Score where a survival benefit from transplant could be reasonably expected for a patient in CR1 in each group (assuming a suitable donor could be found, the patient was willing to undergo transplant, etc). If no patient in a group should be referred for transplant, put N/A. The first table is the master table for all patients (similar to what appears in the Loke, et al, 2023) but with option to put a different threshold depending on patient age/PS. Additional table templates have been provided for specific molecular subgroups (e.g., CBF and NPM1) in case MRD interpretation is likely to be different in these subgroups. If you do fill out the CBF/NPM1 templates then the master table will only be used for non CBF/NPM1 patient. The first table is the master table per ELN risk favorable, intermediate and adverse proving the MRD status (positive and negative) for selective age groupings. The latter tables include patients with CBF leukaemia, and patients with NPM1 mutated disease.

Traditional AI techniques rely on large amounts of clean data, well organized and in a useable format. The system described herein uses approximately 10% of data required for model building versus traditional AI techniques, does not rely on clean datasets and offers explainable outcomes. The model building process is agnostic and does not rely on individually identified datasets. This supports anonymity. As stated above, the system takes in account input from a mixture of researchers, each with expertise in very different areas. As a result, the methodology behind this system focuses on a combination of different data sources and analysis techniques in addition to the standard clinical trial criteria.

This system involves a variety of techniques including both qualitative and quantitative data. This patient analysis emphasizes active engagement in all stages of the investigation, thus empowering patients to ensure the best possible outcome. The system uses a variety of these methods including patient-reported outcomes (“PROs”), a measure of how patients feel about their health and well-being at different stages of treatment, qualitative research methods and participatory research. The system can be applied to patients and their perspectives in the design, implementation, and interpretation, and dissemination.

Data can be collected from subject matter experts (“SMEs”). Following this, with the support of a registry, the required data can be collected in a wider variety of different settings including face-to-face interviews and surveys. Open source toolsets and specialized tools can be used to manage uncertainty and complexity in multiple areas of analysis including data collection and analysis. These are integral for the collection and analysis of messy and incomplete data, which is a key issue in the research of AML.

Mapping (and remapping) the patient journey as an important step in healthcare intervention research and development for several reasons. First, the patient journey map provides a detailed understanding of a patient's experience with a particular health condition from symptom onset, through diagnosis, treatment, and even post-treatment. This detailed, human-centered insight can reveal pain points, needs, and opportunities for interventions that can improve outcomes and the overall experience.

Second, by understanding the patient's journey, the system identifies key decision points where interventions can be applied for model intervention points. These could be preventative measures before the disease onset, early diagnostic tools, effective treatments, or supportive services after treatment. Third, having a clear patient journey map helps to set realistic goals and expectations for the intervention, considering the actual context in which it will be used. It provides a sense of how much improvement can be made, and in which aspects of the patient journey.

Fourth, by considering the patient's perspective and understanding their needs at different stages of the journey, the system design is more effective, usable, and provides appealing solutions. It ensures that interventions are not just theoretically effective, but practically applicable and acceptable to patients. Fifth, understanding the patient journey can inform the development of strategies to increase patient engagement and adherence to treatment. This is important for interventions to be effective in real-world settings. Lastly, a detailed understanding of the patient journey can contribute to developing interventions that improve health outcomes. By targeting the right points in the journey with effective solutions, the system can maximize the impact of the intervention.

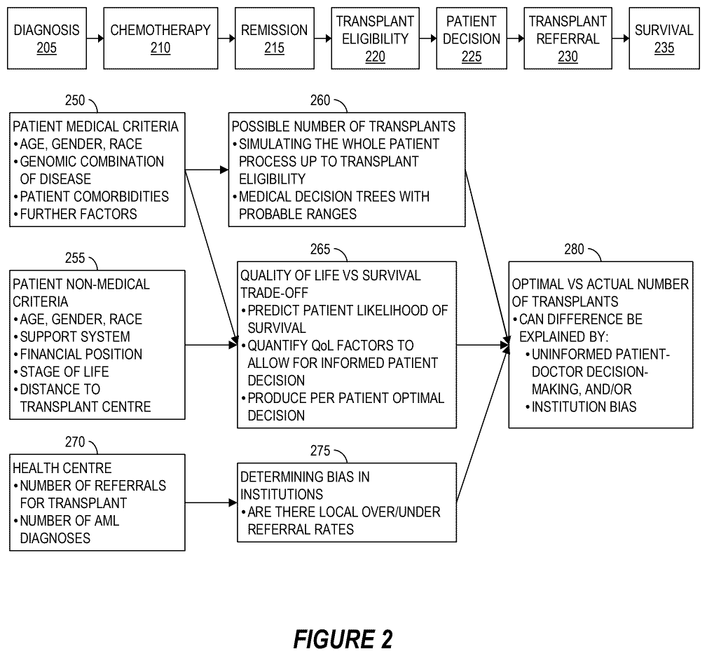

FIG. 2 illustrates a block diagram of an example patent journey. The journey for a patient who is diagnosed with AML is long, complex and full of big decisions. Currently, when diagnosed 205 with AML the most common initial treatment is chemotherapy 210 (there are some cases where no treatment will be given). If after this chemotherapy a patient is in remission 215 they may (given the severity of the disease, comorbidities and the age of the patient) be given the option to have a curative transplant. The option for a curative transplant includes a transplant eligibility analysis 220, a patient decision 225 to pursue the curative transplant, a transplant referral 230 towards survival 235. There are different patient and clinician factors that may impact the decision making process. The decision making process takes into account patient medical criteria 250 and patient non-medical criteria 255. The patient medical criteria 250 is fed into a possible number of transplants 260 and quality of life versus survival tradeoff 265 (which also receives the patient non-medical criteria 255). The decision making process also takes into account heath center considerations 270 including determining biases in institutions 275. The possible number of transplants 260, quality of life versus survival tradeoff 265, and biases in institutions 275 all play a part in the optimal versus actual number of transplants 280.

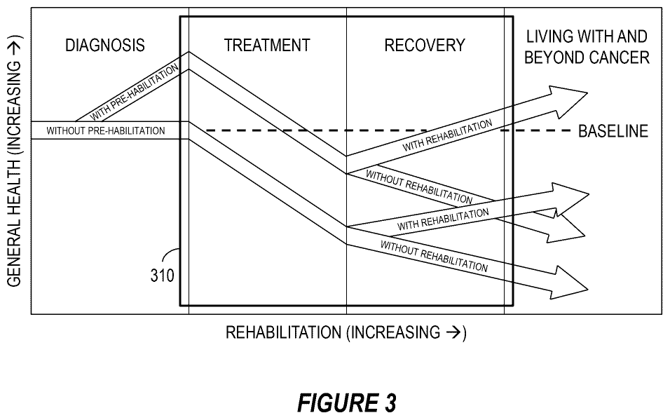

FIG. 3 illustrates a diagram demonstrating the patient journey from diagnosis to living with and beyond cancer. The system addresses region 310 toward a decision to move forward to treatment including the patient decision and creating a toolkit that supports the decision making process.

With respect to data collection, data will come from a mixture of sources including a literature review, surveys, open-source data sources, structured interviews, historical data, subject matter experts, workshops and focus groups. All data collected will be stored in an organized, secure and safe way, in line with all data ethics and privacy guidance. The literature review will be used for establishing the research setting and identifying gaps in the current knowledge base. Initially a literature review provides context, justification of the research question, and potential methodology and design considerations to support the data collection. Other relevant information on supporting data-led patient decision making and the use of AI/ML to enable this.

The surveys will be used to gather information from a sample of individuals including; doctors, patients, the AML working party, patient advocacy groups. Open-source data refers to publicly available information that has been collected and made accessible for research purposes. Data sources are likely to include cancer research, Office of National Statistics (“ONS”) and census data.

Structured interviews can also be used for the data collection. This involves a predetermined, and approved, set of questions that are asked to each participant in a consistent and standardized manner. These questions can be conducted in face-to-face interviews with a variety of different stakeholders including with patients who are at different stages in their AML pathway. Historical data obtained from the AML working party and from clinical trials will be used to populate the model where appropriate, as well as to provide valuable insights into the past events and trends associated with the treatment of AML.

Subject matter experts will be used to provide valuable insights, opinions, and expertise. Initially subject matter expert insights will be used to fill in the data gaps, which will subsequently be filled with data collected. The subject matter experts will also be consulted throughout the process to guide the model build and to assist in the generation of survey questions. Their expertise will also be used to validate research findings and provide contextual information. Workshops and focus groups are used to gain qualitative data and gather in-depth feedback. This feedback can be delivered via guided conversations, open conversations and specific questioning.

The participation criteria centers around ensuring that there is a range of participants. This will consist of anyone who has experience of AML treatment including doctors, nurses, other practitioners, patients currently undergoing AML treatment, patients pre-curative transplant, patients post curative transplant, family of the patients as well as researchers and patient advocacy groups. Ideally there will be a representative split of the demographic, psychographic, sociographic, geographic, ethnographic, physiographic, topographic, chronographic of AML patients that will be statistically significant, and support the driving agenda of equality and equity, as determined by the initial modelling, workshops and literature review. The information will indicate some of the potential areas of interest that can be used to direct future registry questions.

The system uses a predictive decision support platform. The platform can address virtually any problem, particularly those problems with significant analytical and organizational complexity. The system enables the creation of a model that can incorporate and evaluate uncertainty, this means that it is able to cope with incomplete or messy data. The approach to this involves discussion with subject matter experts to identify key points within the data that can be extrapolated. This data collection method allows for distribution within the data, and will account for uncertainty within the data set. These data points are then analyzed using Monte Carlo simulations to predict possible outcomes that will be used to aid decision making.

In addition, different data science and analysis methods are used as appropriate, given the data collected. This will include, but will not be limited to, regression analysis, classification, clustering, network analysis and time series analysis. Data management is employed as well. The system will ensure that robust security measures are implemented, that comply with General Data Protection Regulation (“GDPR”) in the European Union. All participants will be appropriately consented. Data collection and storage are secure, reliable and scalable. As per the NHS guidelines all of this information will be accessible, the system also ensures that the AML working party have access to findings as well as the model itself (see TABLES 3-5 above).

There are a number of factors that will be considered including informed consent, privacy and confidentiality, fairness, data analysis transparency and AI and machine learning processes and algorithms. Informed consent is essential for all participants, and ensures they understand the purpose, risks, benefits, and potential outcomes of the study. Participants will have the right to refuse participation or withdraw from the study at any time without penalty. Privacy and confidentiality is key to safeguarding patient data. All data collected will be stored and used in a way in which identifiable information is accessed only by authorized personnel. All data will be anonymized and patient privacy will be protected unless explicit consent is obtained for the use of identifiable data such as disclosed in U.S. patent application Ser. No. 19/000,364 introduced above.

All participants will be treated in a fair and equitable manner, ensuring respect and inclusivity. Approval for all questions will be sought from the appropriate ethics review board or committee. Data analysis transparency is a value that is key to the system. The ‘glass box’ and low code/no code modelling approach makes this accessible. This maintains transparency throughout the research project by accurately representing the goals, methods, and outcomes. AI and Machine Learning processes and algorithms may be used in which case, the utilization of all data will be controlled in an ethical and unbiased way. Any issues of algorithmic fairness, transparency, and accountability will be addressed immediately.

Engagement with patients and patient advocacy groups will be key. As such, there are a number of steps that will be taken to ensure this including communication strategy, dissemination in the medical and patient community, and finally, monitoring and evaluation of feedback. A communication strategy with the involved stakeholders defines the objectives and desired outcomes for the dissemination effort. This will include multiple visual aids such as reports, presentations, infographics, and graphs. These will be tailored to the target audience.

Monitoring and evaluation of the feedback to the system is useful to address concerns clarify any ambiguities, and provide additional information as needed to ensure accurate interpretation and understanding of the research findings. This should be done for both the medical and patient communities to ensure equity in outcomes and quality of life for all patients.

The system provides a ‘toolkit’ for improving patient understanding and decision making regarding elective treatment. This will be used within the AML patient and doctor community. It is important to note that while this system focuses on AML, the underpinning model is based on the understanding of patient decision making, which is applicable across a wide range of illnesses and diseases.

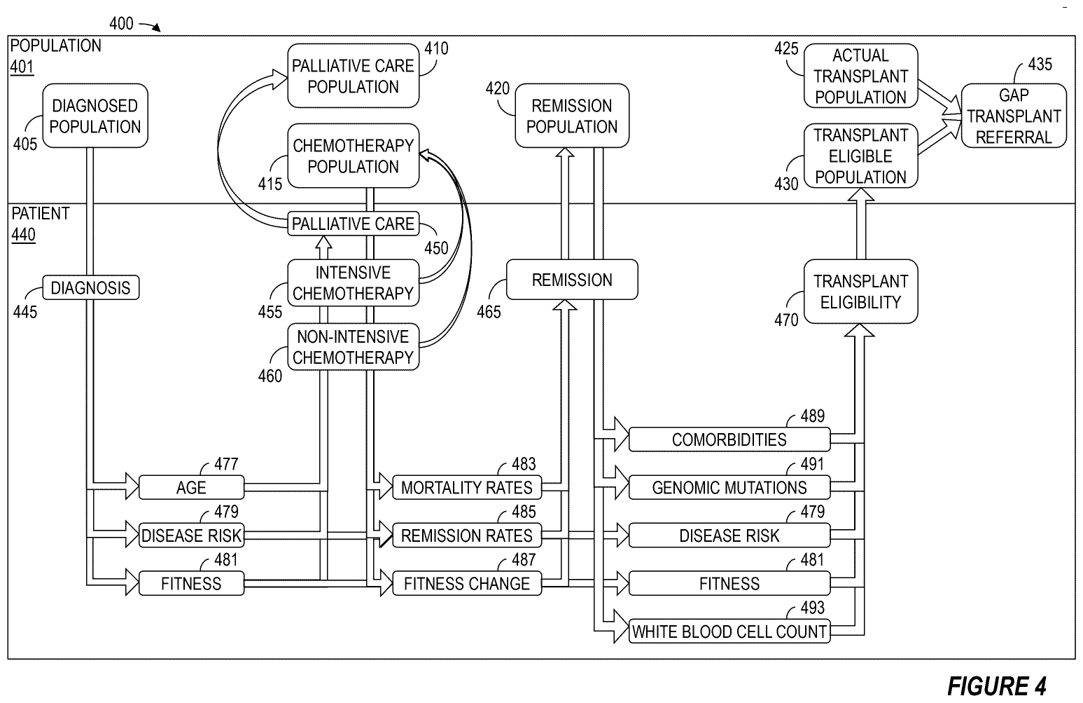

FIG. 4 illustrates a block diagram of a system to predict and prescribe treatments and remedies for diseases in the healthcare industry. The system includes a population and patient model 400 that takes into account population data 401 and patient factors 440. The population data 401 include the diagnosed population 405, palliative care population (a population of patients under different treatments) 410 and chemotherapy population (a population of patients under different treatments) 415, remission population (a cured population) 420, actual transplant population (actual treatment population) 425, transport eligible population (treatment eligible population) 430 leading to a gap in transport referral 435 between the actual treatment population 425 and the treatment eligible population 430. The patient factors 440 include a patient diagnosis 445, palliative care (a patient treatment option) 450, intensive chemotherapy (a patient treatment option) 455, non-intensive chemotherapy (a patient treatment option) 460, remission (a cured patient) 465 and transplant eligibility (a treatment) 470. The patient factors 440 also take into account characteristics such as age 477, disease risk 479, fitness 481, mortality rates 483, remission rates 485, fitness change 487, comorbidities 489, genomic mutations 491 and white blood cell counts 493.

The diagnosed population 405 feeds into the patient diagnosis 445 and takes into account age 477, disease risk 479 and fitness 481 as input to the patient palliative care 450, intensive chemotherapy 455, and non-intensive chemotherapy 460. If palliative care 450 is the option then that is added to the palliative care population 410. If intensive chemotherapy 455 or non-intensive chemotherapy 460 are the options then that is added to the chemotherapy population 415. The palliative care population 410 and the chemotherapy population 415 take into account mortality rates 483, remission rates 485, fitness change 487 as an input to the patient remission 465, which is added to the remission population 420. The remission population 420 takes into account comorbidities 489, genomic mutations 491, disease risk 479, fitness 481, and white blood cell counts 493 as a input to the patient transplant eligibility 470, which is added to the transport eligible population 430. Comparison of the actual transplant population 425 and transport eligible population 430 provides the gap in transport referral 435.

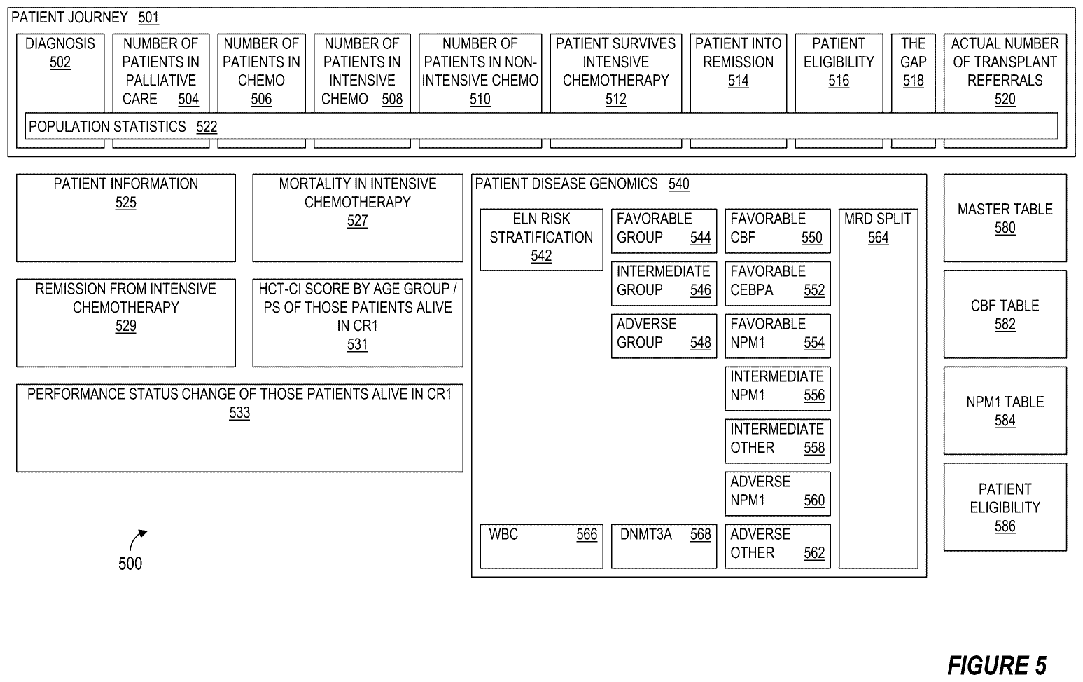

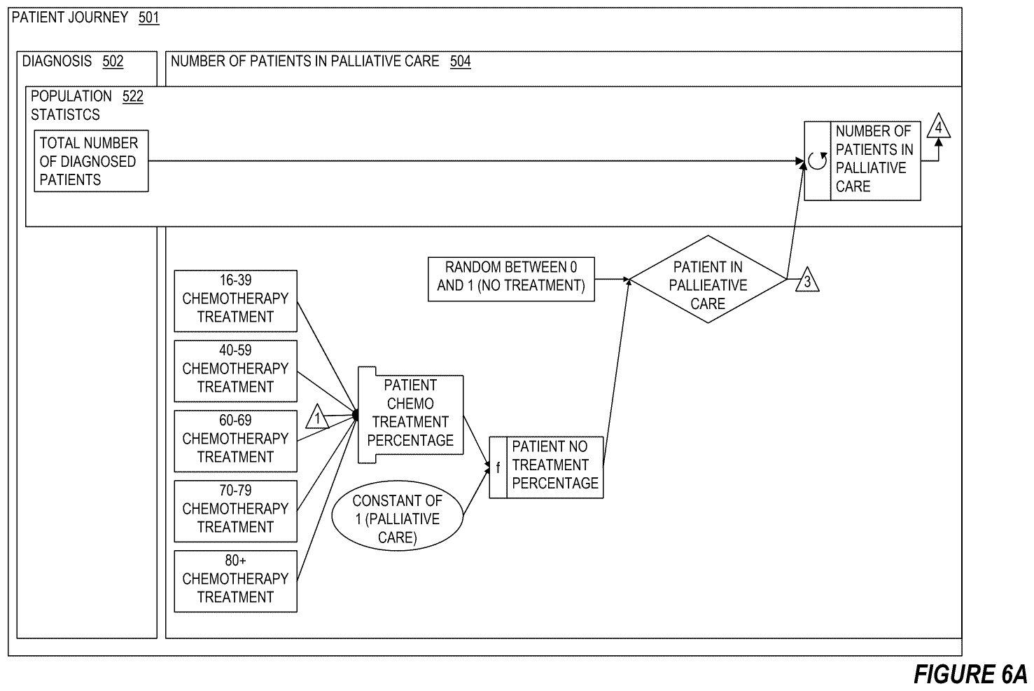

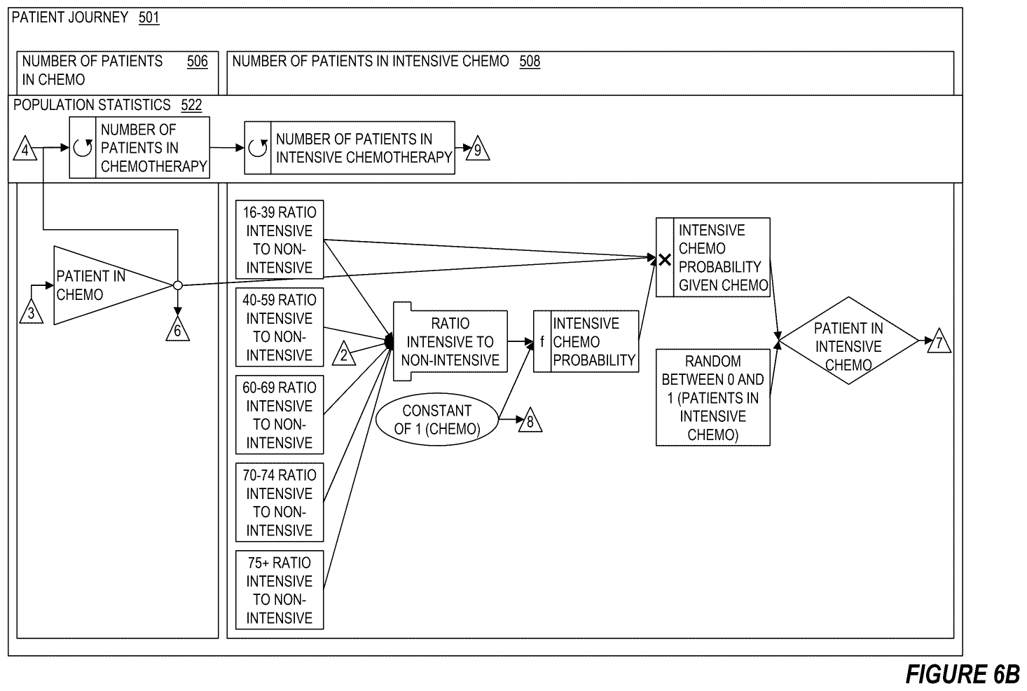

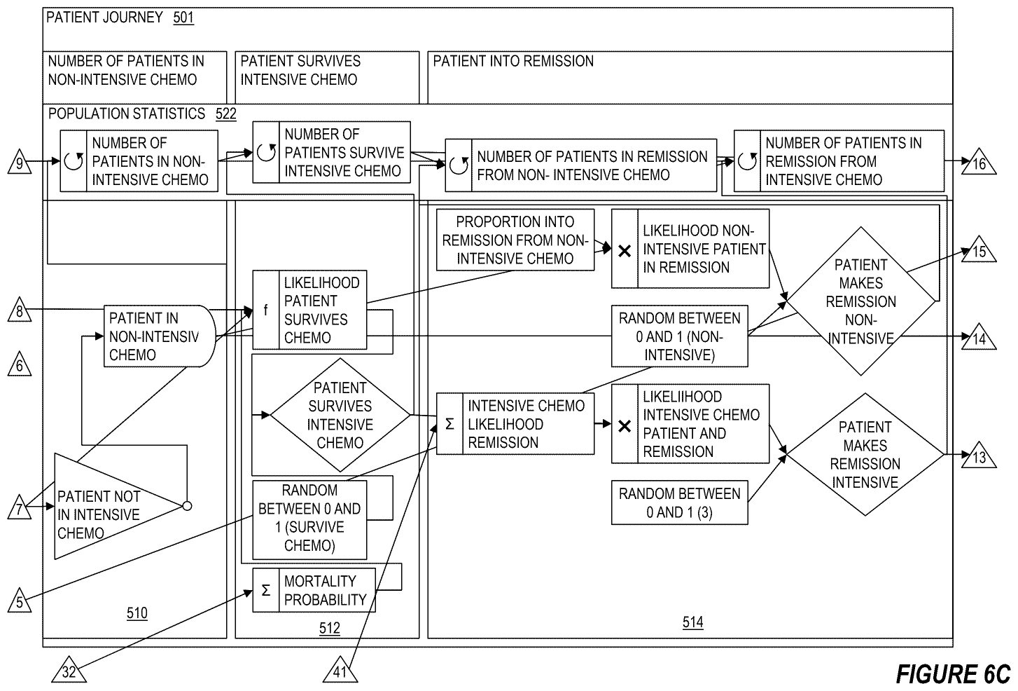

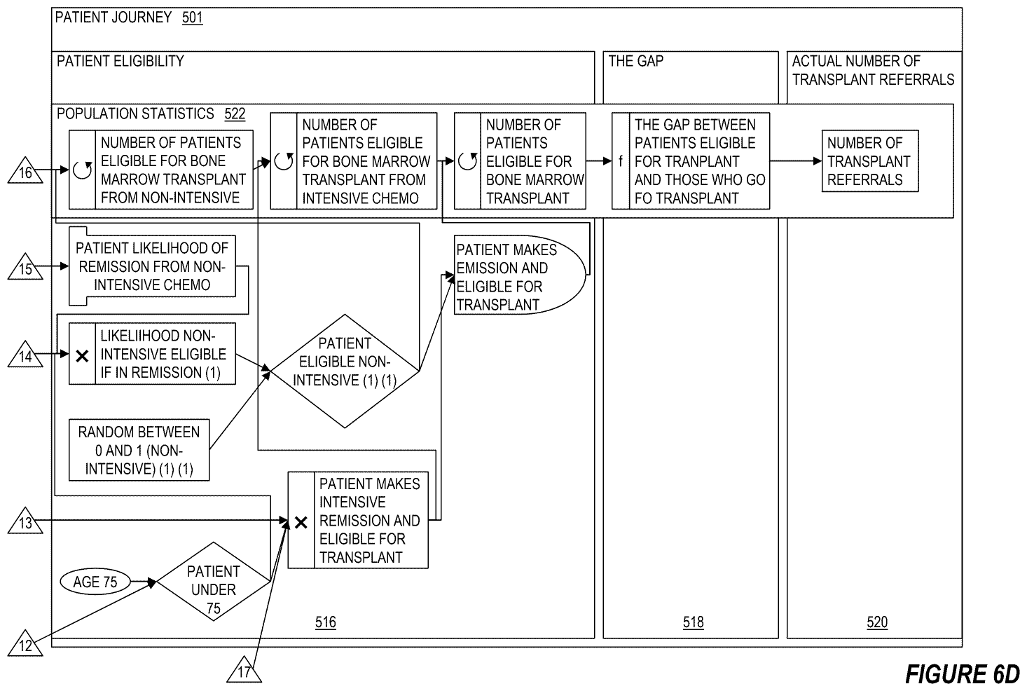

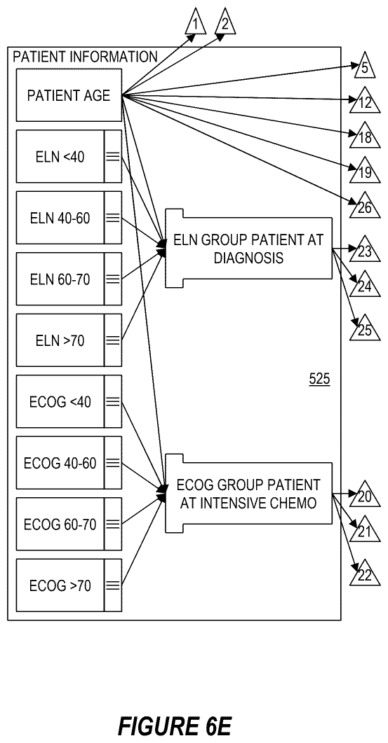

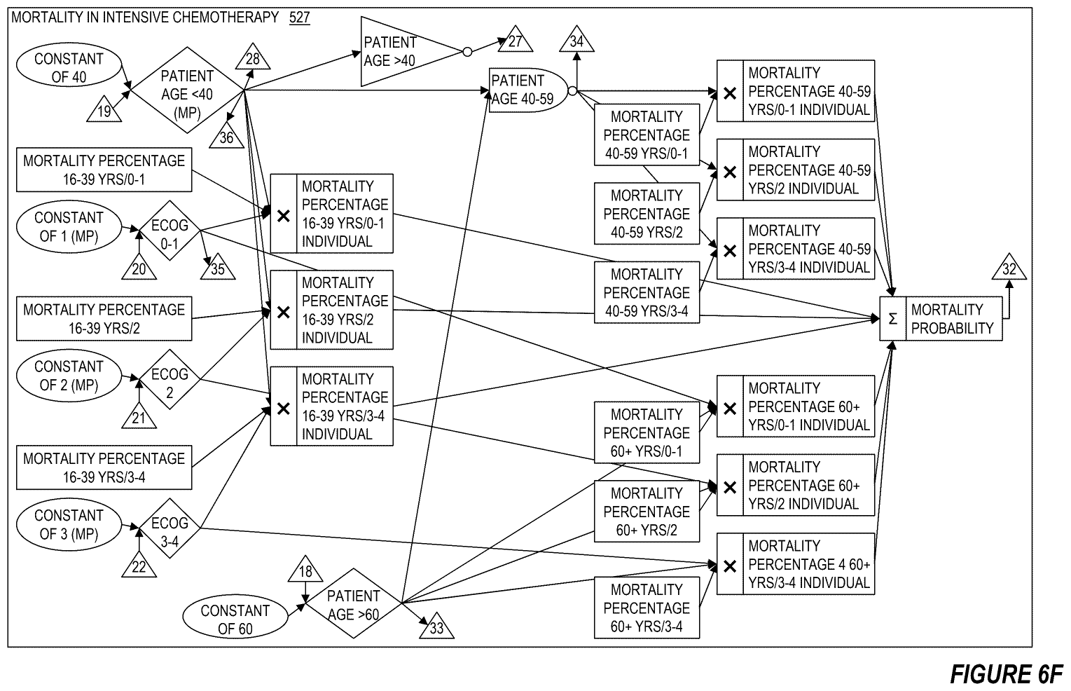

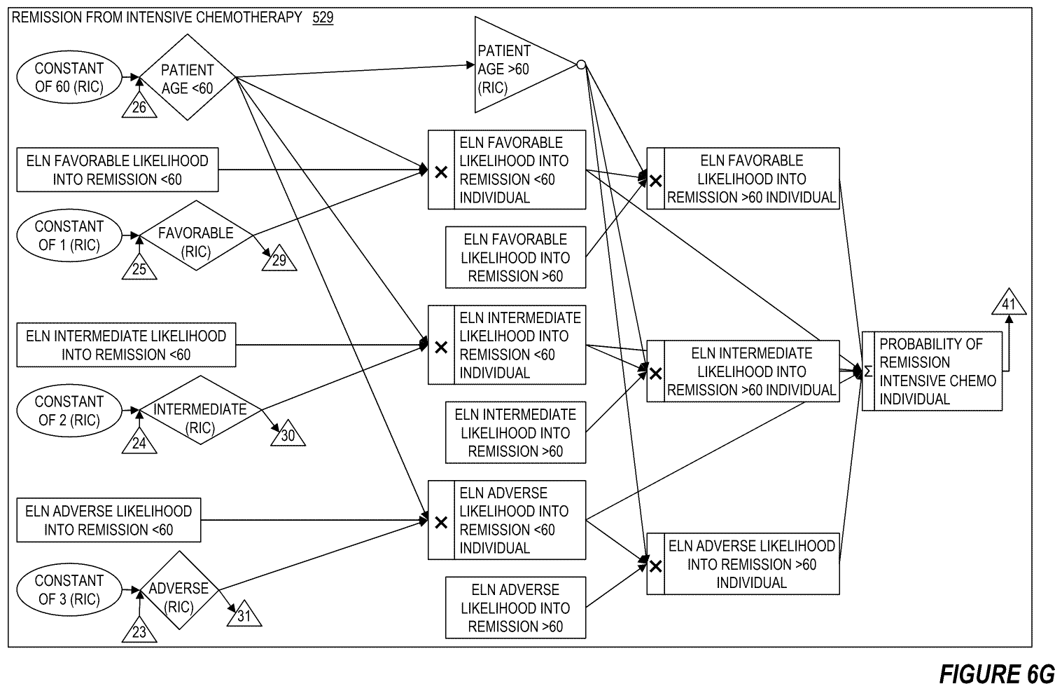

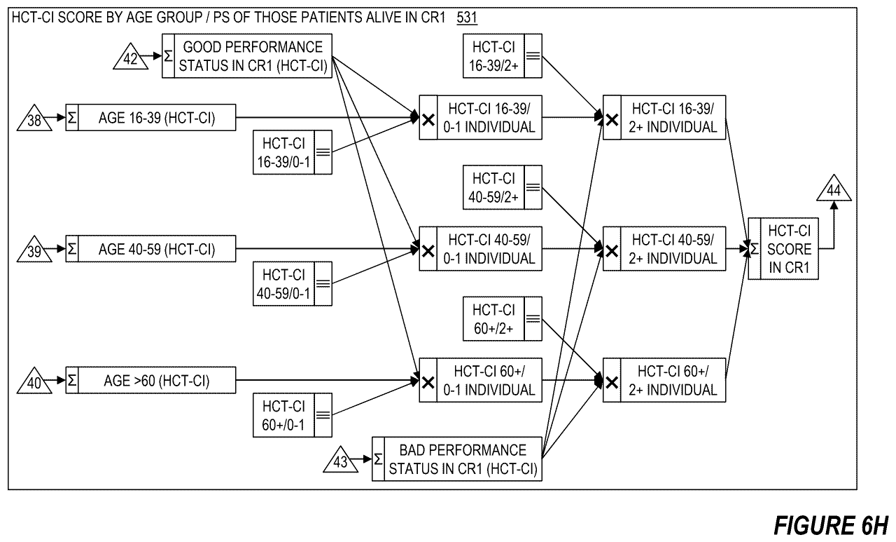

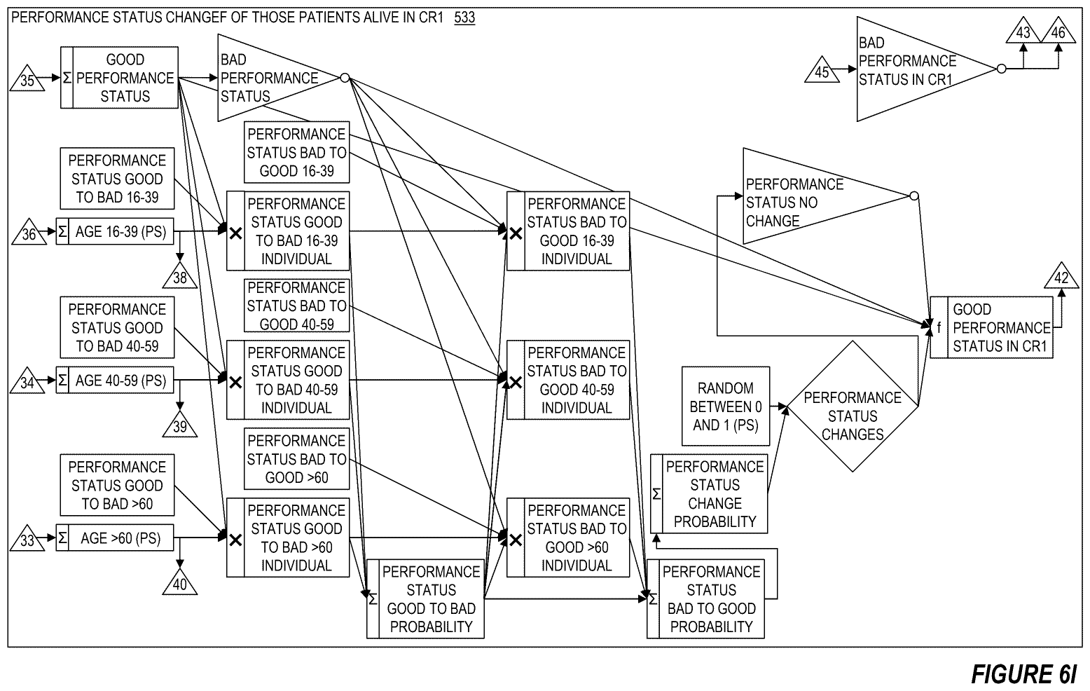

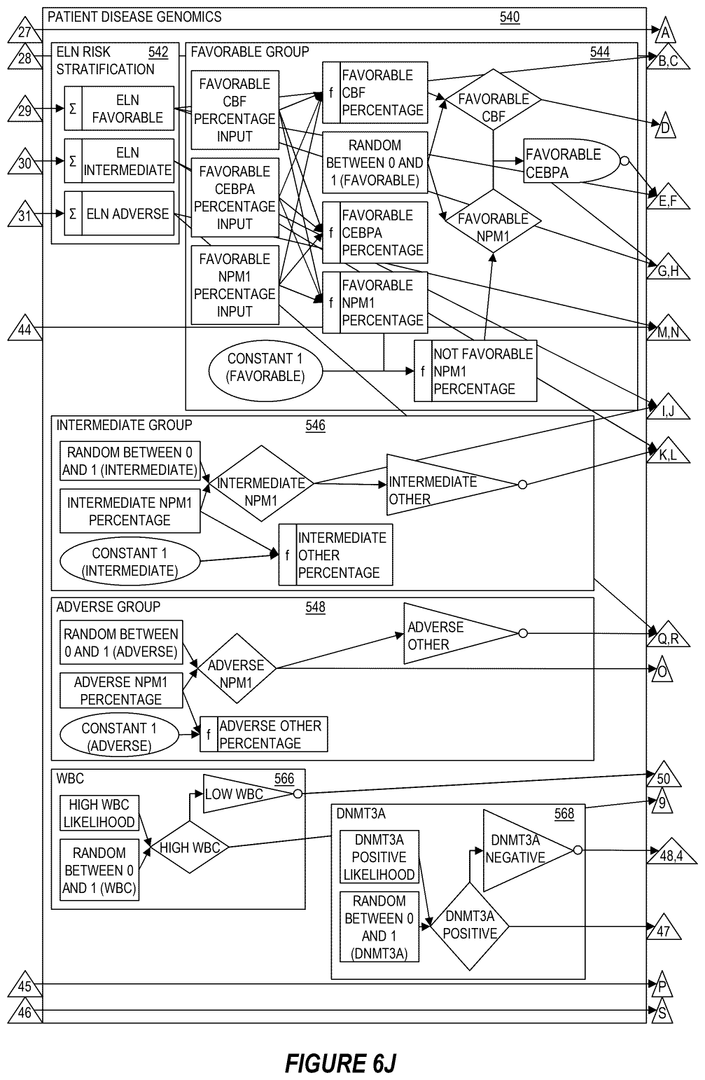

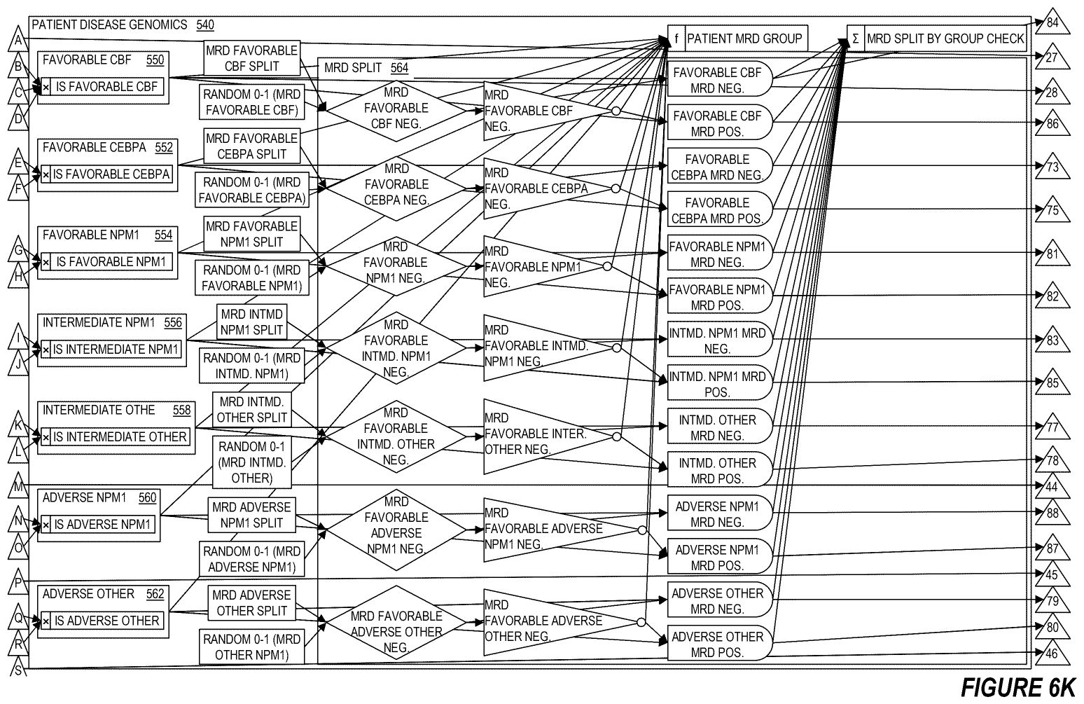

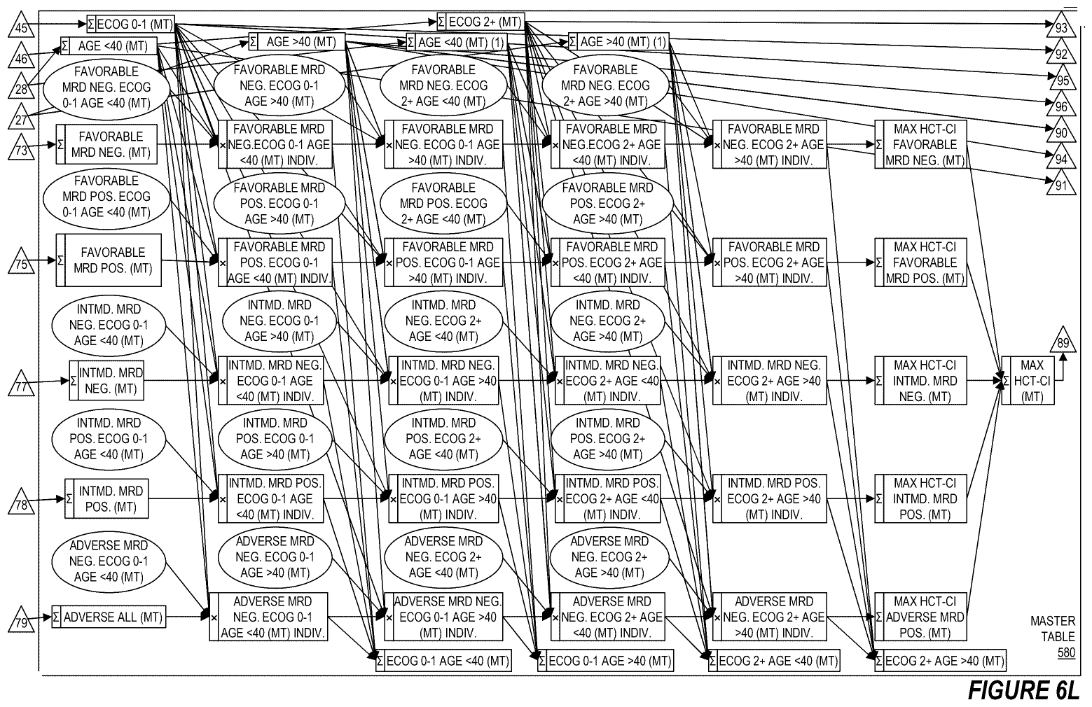

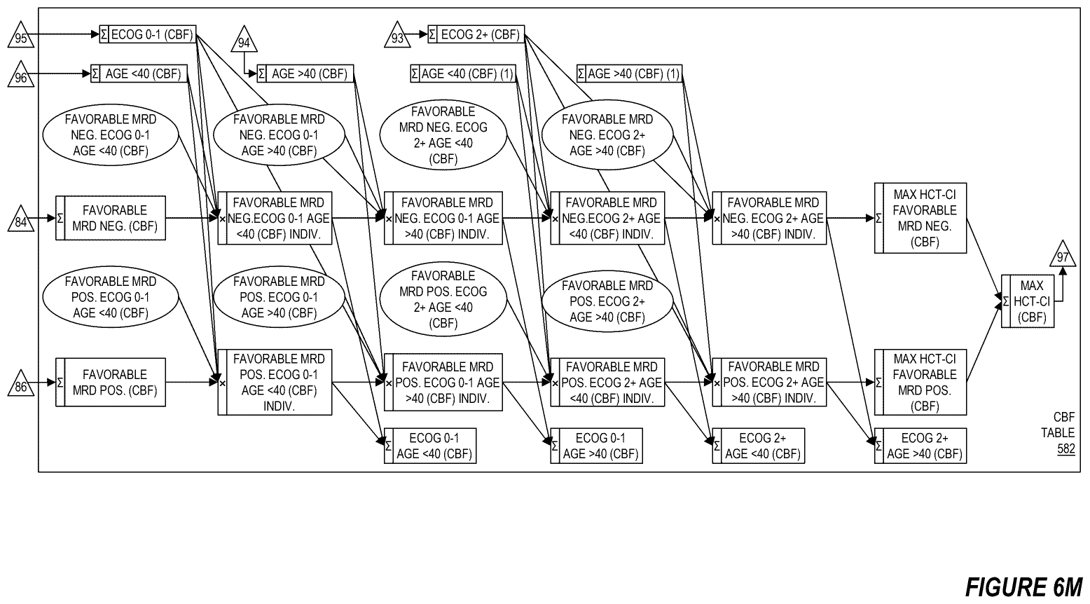

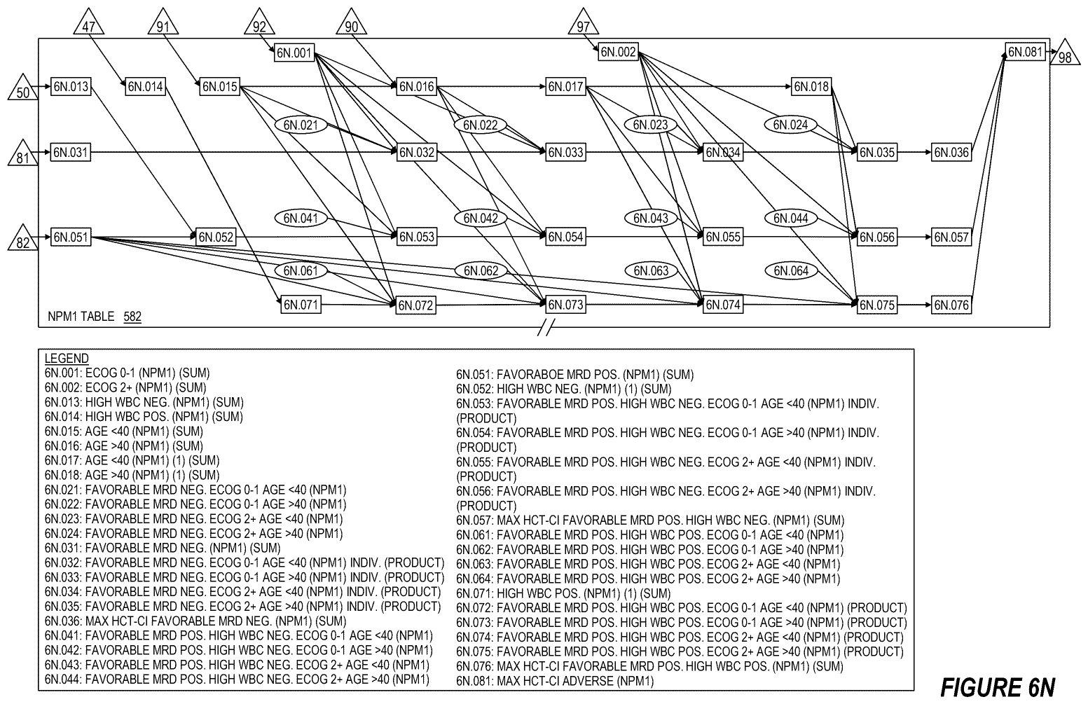

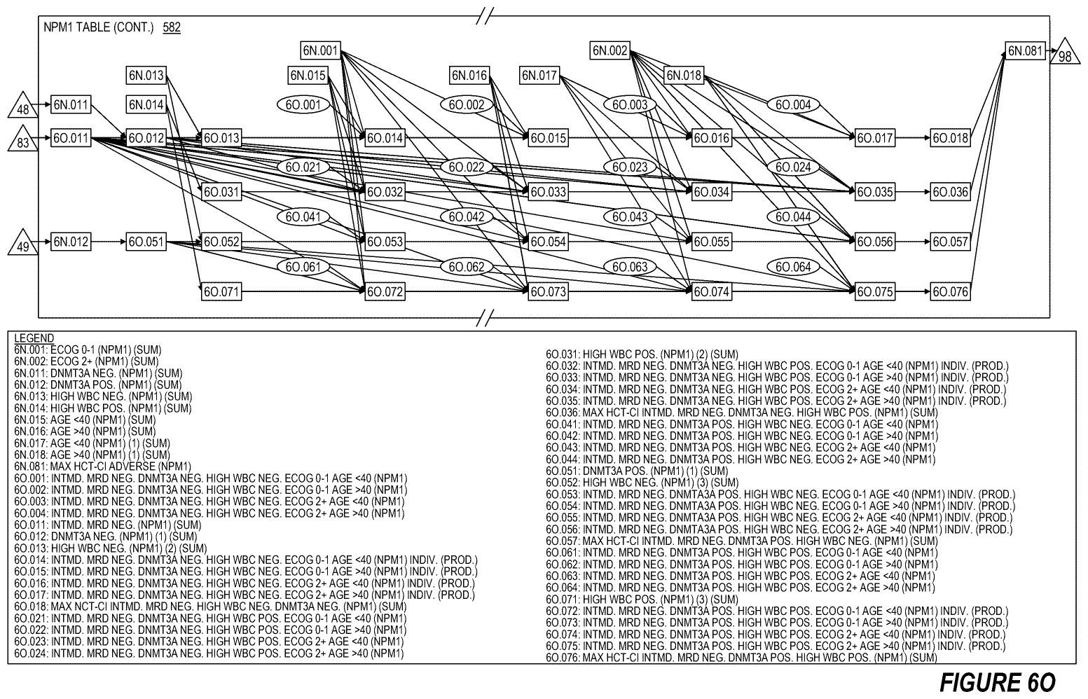

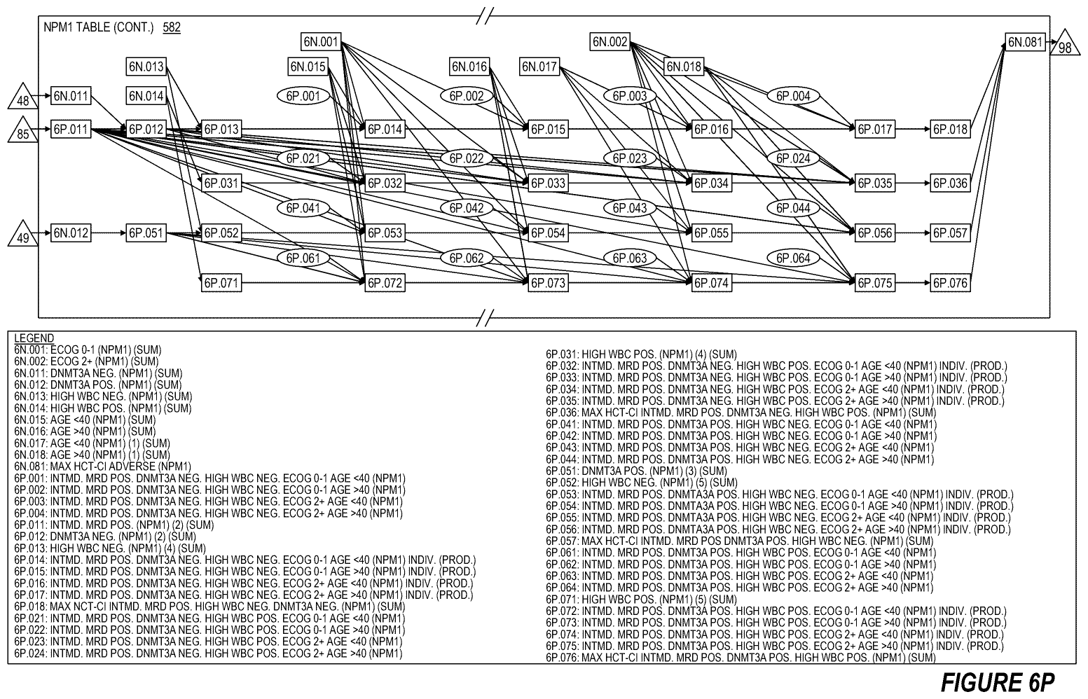

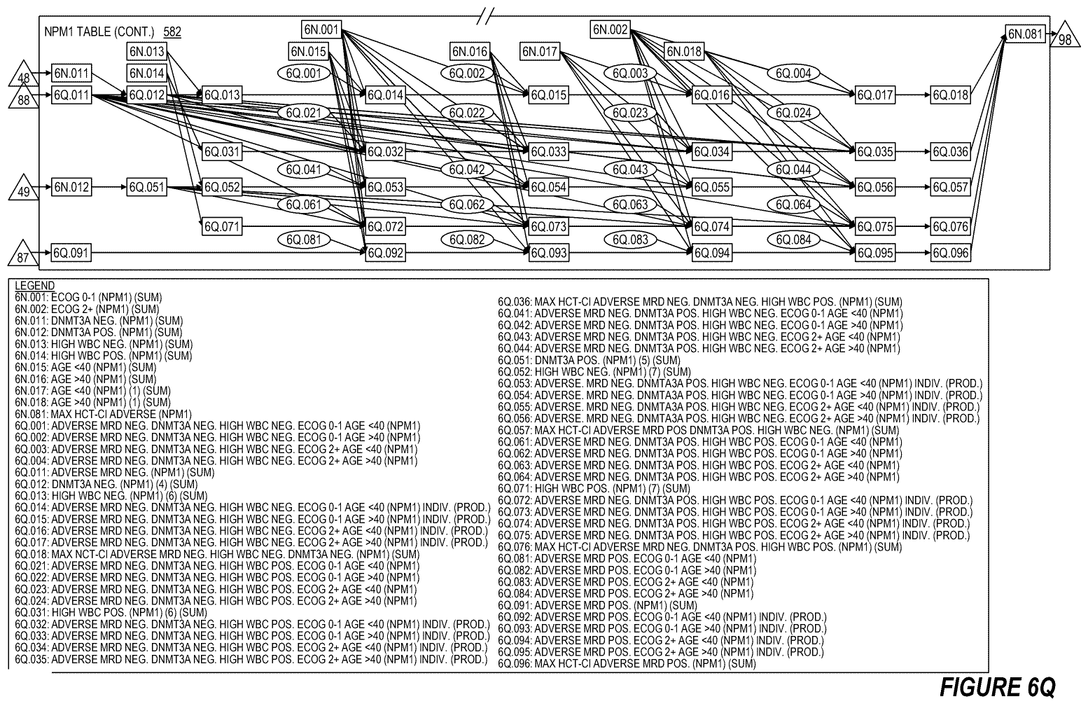

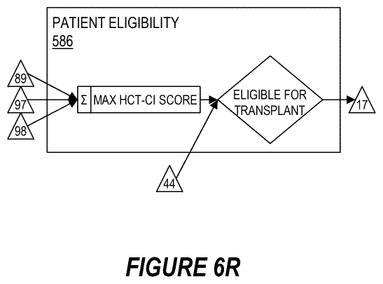

FIG. 5 illustrates a block diagram of a system to predict and prescribe treatments and remedies for diseases in the healthcare industry. FIGS. 6A to 6R illustrate exploded views of the sections of the system of FIG. 5. The system includes a population and patient model 500 including a patient journey 501 that describes the overall clinical pathway of an AML patient, from diagnosis 502 to transplant referral 520 (see also FIGS. 6A to 6D). It also includes predicted and actuals of the population. The diagnosis 502 is the starting point, when a patient is first diagnosed with AML. This contains the number of diagnosed patients per year (population data). The number of patients in palliative care (a population of patients under different treatments) 504 determines whether a patient will receive palliative (comfort-focused care) rather than end of life treatment. The number of patients in chemo (a population of patients under different treatments) 506 shows whether a patient received chemotherapy. The number of patients in intensive chemo (a population of patients under different treatments) 508 determines based on patient age the likelihood of them receiving more invasive intensive chemotherapy or less invasive non-intensive chemotherapy. The number of patients in non-intensive chemo (a population of patients under different treatments) 510 shows whether a patient receives non-intensive chemotherapy. The patient survives intensive chemotherapy 512 determines the likelihood a patient undergoing intensive chemotherapy survives the treatment based on patient age and fitness. The patient into remission (a cured patient) 514 is for patients undergoing non-intensive chemotherapy and determines whether the patient makes it into remission based on probability of making it into remission. For patients undergoing intensive chemotherapy this determines whether they make it into remission based on patient age and disease categorization into favorable, intermediate, or adverse by European LeukemiaNet (“ELN”) risk classification. The patient eligibility (a treatment) 516 determines a patient's eligibility for haematopoietic stem-cell transplantation (“HSCT”), based on three patient eligibility tables below. The gap 518 determines the difference between the predicted number of patients eligible for HSCT (treatment eligible population) and the actual number who receive a curative transplant (actual treatment population). This contains the target, whether the U.K. National Health Service (“NHS”) is under or over referring patients for transplant. The actual number of transplant referrals (actual treatment population) 520 holds actual population level statistics of number of patients per annum who receive a curative transplant. The population statistics 522 holds all the population data, running through all of the above sections sampling from thousands of individually simulated patients.

The patient characteristics and intensive chemotherapy treatment or patient factors (see also FIGS. 6E to 61) includes patient information 525 that contains patient information such as age at diagnosis, ELN risk classification at diagnosis, and ECOG fitness status for patients in intensive chemotherapy. The mortality in intensive chemotherapy 527 determines the likelihood of a patient surviving intensive chemotherapy based on a patients age and ECOG fitness status. The remission from intensive chemotherapy 529 determines the likelihood of a patient who has survived intensive chemotherapy to make it into remission based on a patient's ELN risk classification and age. Based on patient age and performance status (PS-‘good’ or ‘bad’ based on ECOG fitness status), the HCT-CI Score by Age group/PS of those patients alive in CR1 531, this determines the likelihood of a patient having a specific HCT-CI score. Based on a patient's age and performance status, the Performance Status change of those patients alive in CR1 533 determines the likelihood of performance status of a patient in remission changing during the intensive chemotherapy, i.e., from ‘good’ to ‘bad’ or ‘bad’ to ‘good’.

The Patient Disease Genomics 540 (further patient factors) categorises in more detail the disease genomics for each patient in remission, from which eligibility for the curative transplant will be determined (see also FIGS. 6J to 6K). The ELN Risk Stratification section 542 carries through a patient's ELN risk classification (favorable, intermediate, or adverse). The Favorable Group 544 is for a patient in remission with favorable ELN risk, and determines the type of AML the patient has, split into three categories: Core Binding Factor (“CBF”), CCAAT-Enhancer-Binding Protein Alpha (“CEBPA”), and Nucleophosmin 1 (“NPM1”). The Intermediate Group 546 is for a patient in remission with intermediate ELN risk, and determines the type of AML the patient has, split into two categories NPM1 (as above) and Other (representing all not in category NPM1). The Adverse Group 548 is for a patient in remission with adverse ELN risk, and determines the type of AML the patient has, split into two categories NPM1 (as above) and Other (representing all not in category NPM1). The Favorable CBF 550 indicates whether a patient in remission has favourable ELN risk category CBF. The Favorable CEBPA 552 indicates whether a patient in remission has favourable ELN risk category CEBPA. The Favorable NPM1 554 indicates whether a patient in remission has favourable ELN risk category NPM1. The Intermediate NPM1 556 indicates whether a patient in remission has intermediate ELN risk category NPM1. The Intermediate Other 558 indicates whether a patient in remission has intermediate ELN risk category Other. The Adverse NPM1 560 indicates whether a patient in remission has adverse ELN risk category NPM1. The Adverse Other 562 indicates whether a patient in remission has adverse ELN risk category Other. For each of the risk classification/disease genomic categories above, the MRD Split 564 determines whether a patient is minimal residual disease (“MRD”) positive or negative. When a patient is in remission, they receive an MRD test to determine whether there is a presence of a small number of cancer cells in the body. The WBC 566 determines whether a patient has a high white blood cell (“WBC”) count, which is associated with more negative outcomes from the curative transplant. The DNMT3A 568 determines whether a patient has a mutation in DNA Methyltransferase 3A (“DNMT3a”) gene. DNMT3a mutations have been associated with adverse prognosis for AML patients in general but their effect on the outcome of HSCT is not fully understood.

Based on a patient's disease genomics, eligibility for transplant is determined by three separate tables (see also FIGS. 6L to 6Q). Each table looks at a patient's age, ECOG fitness status, MRD status, and ELN risk status, white blood cell count, DNMT3a mutation status, to determine the maximum HCT-CI score for which a patient would be considered eligible for the curative transplant. The Master Table 580 combines the above information to determine maximum HCT-CI score for which a patient would be considered eligible for transplant for risk classification/disease genomic categories, Favorable CEBPA, Intermediate Other, and Adverse Other. The CBF Table 582 combines the above information to determine maximum HCT-CI score for which a patient would be considered eligible for transplant for risk classification/disease genomic categories Favorable CBF. The NPM1 Table 584 combines the above information to determine maximum HCT-CI score for which a patient would be considered eligible for the curative transplant for risk classification/disease genomic categories, Favorable NPM1, Intermediate NPM1, and Adverse NPM1. The Patient Eligibility (a treatment) 586 takes the maximum HCT-CI score for which a patient would be considered eligible for transplant and compares it with the patients HCT-CI score to determine whether the patient is eligible for the curative transplant or not (see also FIG. 6R).

Thus, the inputs to the model include, without limitation:
Age Spread,
ELN Risk Classification Spread by Age,
HCT-CI Score Spread by Age,
ECOG Fitness Score,
Percentage of patients who do not go to chemotherapy,
Ratio of Intensive to Non-Intensive Patient by Age,
Proportion of Patients into Remission from Non-Intensive Chemotherapy,
Likelihood into remission from Intensive Chemotherapy by ELN Risk,
Performance Status change during Intensive Chemotherapy,
Favorable (Core Binding Factor (“CBF”), CCAAT Enhancer Binding Protein A (CCAAT: cytosine-cytosine-adenosine-adenosine-thymidine) (“CEBPA”), Nucleophosmin 1 (“NPM1”)), Intermediate (NPM1, Other), Adverse (NPM1, Other) Group Split,
MRD Negative Split by each group, and
CBF, NPM1, Loke, et al., 2023 lookup tables to determine eligibility.

The results of model employing the model inputs are summarized in TABLE 6.

TABLE 6
Model Outputs

| | | | | | | | | | Could | |
|---|---|---|---|---|---|---|---|---|---|---|
| | | | | | Survive | Into | Eligible for | | have | |
| | | Diagnosed | In Chemo | | Chemo | Rem | BMT | | BMT | BMT |
| N-IC | | | | 1053 | | 479 | 153 | | | |
| | | | | 1029 | | 460 | 141 | | | |
| | | | | 1013 | | 447 | 123 | | | |
| | | | | 995 | | 435 | 115 | | | |
| | | | | 972 | | 416 | 108 | | | |
| Pop | PH | 3022 | 2325 | | | | | 648 | 330 | 318 |
| | LH | 3022 | 2301 | | | | | 620 | 302 | 318 |
| | Nom | 3022 | 2287 | | | | | 569 | 251 | 318 |
| | LL | 3022 | 2273 | | | | | 503 | 185 | 318 |
| | PL | 3022 | 2248 | | | | | 483 | 165 | 318 |
| IC | | | | 1317 | 1200 | 792 | 555 | | | |
| | | | | 1292 | 1175 | 768 | 531 | | | |
| | | | | 1276 | 1159 | 751 | 436 | | | |
| | | | | 1258 | 1142 | 736 | 393 | | | |
| | | | | 1231 | 1114 | 714 | 376 | | | |

*Where “Pop” refers to Population, “PH” refers to Possibly High, “LH” refers to Likely High, “Nom” refers to Nominal, “LL” refers to Likely Low, “PL” refers to Possibly Low, “Chemo” refers to Chemotherapy, and “Rem” refers to Remission.
Also, while the model and results refer to bone marrow transplant, any type of curative transplant (a treatment) may be employed.

The results prove that there is a gap between current eligibility for the curative transplants and possible eligibility. Based on the exemplary output above, a median of an additional 251 patients per year could have received a curative transplant. In general, the model predicts patients eligible for transplant is up to 50-100% higher than observed transplantations. The model allows for running scenarios per patient. One Monte Carlo analysis is performed per patient, then an iterator can be used to estimate population spread. The models generate specific insights for individual patients, with each simulation representing one synthetic patient. Though each simulation represents an individual patient, the modeling can scale to answer complex questions about population health based on, for instance, thousands of individual patient journeys. By simulating a patient's entire care pathway, clinicians can make more informed decisions about treatment plans, and customize interventions to each patient's specific need. The models support healthcare management teams, assisting cancer alliances and healthcare trusts by identifying optimal resource allocation to improve overall patient outcomes. Of course, the method could be applied to other diseases or to answer other issues within healthcare.

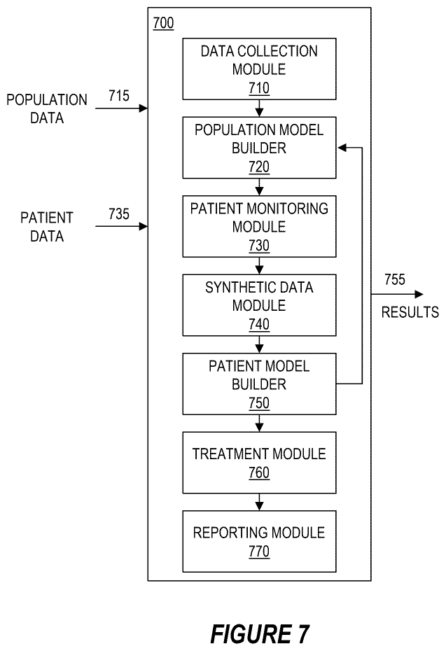

FIG. 7 illustrates a block diagram of a system 700 to predict and prescribe treatments and remedies for diseases in the healthcare industry. The system 700 includes a data collection module 710 to collect population data 715 about the disease such as AML. As described above, the population data 715 will come from a mixture of sources including a literature review, surveys, open-source data sources, structured interviews, historical data, subject matter experts, workshops and focus groups. All data collected will be stored in an organized, secure and safe way, in line with all data ethics and privacy guidance. In the case of AML, the data collection and analysis may include interviews with doctors, nurses, hematologists, transplant doctors to design questions for patients and patient advocacy groups. Once the questions are answered, data is analyzed and missing data is captured.

The population data 715 includes, without limitation, a diagnosed population, a population of patients under different treatments, cured population, actual treatment population, and treatment eligible population. In the case of AML, the population data 715 includes, without limitation, diagnosed population, palliative care population, chemotherapy population, remission population, actual curative transplant population, and curative transport eligible population.

The system 700 includes a population model builder 720 to build a population model as part of a population and patient model to determine likely impacts of existing or new constraints and interventions. In addition, different data science and analysis methods are used as appropriate, given the data collected. This will include, but will not be limited to, regression analysis, classification, clustering (see, e.g., U.S. patent application Ser. No. 18/475,963 introduced above), network analysis and time series analysis. These data points are then analyzed using Monte Carlo simulations to predict possible outcomes that will be used to aid decision making.

The system 700 includes patient monitoring module 730 to record patient factors 735 of the patient associated with the disease. The patient factors 735 include, without limitation, patient diagnosis, patient treatment options, a cured patent, age of the patient, disease risk for the patient, fitness of the patient, and mortality rates. In the case of AML, the patient factors 735 include, without limitation, patient diagnosis, patient palliative care option, patient intensive chemotherapy option, patient non-intensive chemotherapy option, patient remission, age of the patient, disease risk for the patient, fitness of the patient, and mortality rates.

The patient monitoring module 730 can engage patients in healthcare monitoring and management, using sensors, for instance, wearables or mobile devices to record patient factors 735 such as clinical data including the fitness, vitals, etc. of the patient, complete longitudinal surveys or free-text logs (see, e.g., U.S. Pat. No. 12,013,680 introduced above). The system 700 also includes a synthetic data module 740 to anonymize the patient factors or patient data 735 (see, e.g., U.S. patent application Ser. No. 19/000,364 introduced above) and patient privacy will be protected unless explicit consent is obtained for the use of identifiable data. All data collected will be stored in an organized, secure and safe way, in line with all data ethics and privacy guidance.

The system 700 includes a patient model builder 750 to merge the patient factors 735 into the population and patient model (as indicated by the arrow into the population model builder 720). The system 700 develops a comprehensive data driven model that captures patient-reported expected outcomes for elective treatment, and collects data on patient-reported actual outcomes following elective treatment. In accordance therewith, the system 700 includes a treatment module 760 to prescribe a treatment (or remedy) to the disease for the patient based on predicted outcomes from the population and patient model. The system 700 takes into account patient medical factors or criteria and patient non-medical factors or criteria in prescribing a treatment. The system 700 compares and analyzes the expected versus actual outcomes to determine any disparities, and predicts the degree to which the factors impact treatment decisions.

The system 700 creates a predictive framework (results oriented-prescribing an action based on an application of the model and data to a disease of a patient) to determine where changes are likely to have the biggest impact on positive patient outcomes, leading to supporting prioritized supply chain monitoring. The system 700 enables discovery research and development of new therapeutics (application of remedies to diseases-prescribing an action based on an application of the model and data to a disease of a patient). The system 700 optimizes patient pathway decisions and new intervention options into the patient pathway. There are patients that could receive better outcomes if there was a framework that accounted for the trade-offs between the quality of life and mortality, in terms of the physiological, psychological, social, and economic factors at play. (See, e.g., U.S. Pat. No. 12,013,680 introduced above.)

The system 700 includes a reporting module 770 to report per-patient and per-population results (including the treatment) 775 to a database of, for instance, the respective agencies and healthcare professionals. For example, the system 700 identifies AML population statistics and maps the whole process from diagnosis, to chemotherapy, to patient transplant eligibility. The system 700 can generate and report the difference (a gap) between the actual treatment population and a treatment eligible population to refine the population and patient model. The reporting module 770 also stores the per-patient and per-population results (including the treatment) 775 to memory such as a memory of the apparatus operating the system or external memory (such as the database). While the diagram demonstrates a flow, selected modules (and steps) may be reordered, repeated, combined, added or omitted depending on the system 700 requirements and the disease under consideration.

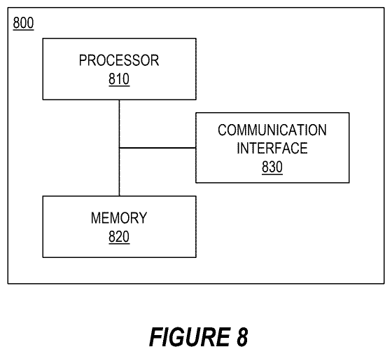

FIG. 8 illustrates a block diagram of an embodiment of an apparatus 800 for operating systems and processes described herein. The apparatus 800 includes a processor (or processing circuitry) 810 a memory 820 and a communication interface 830 such as a graphical user interface. The apparatus 800 operates the tool and system to create predictive models including synthetic datasets and clusters applied to a patient and the population to create remedial measures for of complex diseases such as AML.

The functionality of the apparatus 800 may be provided by the processor 810 executing instructions stored on a computer-readable medium, such as the memory 820 shown in FIG. 8. Alternative embodiments of the apparatus 800 may include additional components (such as the interfaces, devices and circuits) beyond those shown in FIG. 8 that may be responsible for providing certain aspects of the device's functionality, including any of the functionality to support the solution described herein.

The processor 810 (or processors), which may be implemented with one or a plurality of processing devices, perform functions associated with its operation including, without limitation, performing the operations of the systems and processed herein. The processor 810 may be of any type suitable to the local application environment, and may include one or more of general-purpose computers, special purpose computers, microprocessors, digital signal processors (“DSPs”), field-programmable gate arrays (“FPGAs”), application-specific integrated circuits (“ASICs”), and processors based on a multi-core processor architecture, as non-limiting examples.

The processor 810 may include, without limitation, application processing circuitry. In some embodiments, the application processing circuitry may be on separate chipsets. In alternative embodiments, part or all of the application processing circuitry may be combined into one chipset, and other application circuitry may be on a separate chipset. In still alternative embodiments, part or all of the application processing circuitry may be on the same chipset, and other application processing circuitry may be on a separate chipset. In yet other alternative embodiments, part or all of the application processing circuitry may be combined in the same chipset.

The memory 820 (or memories) may be one or more memories and of any type suitable to the local application environment, and may be implemented using any suitable volatile or nonvolatile data storage technology such as a semiconductor-based memory device, a magnetic memory device and system, an optical memory device and system, fixed memory and removable memory. The programs stored in the memory 820 may include program instructions or computer program code that, when executed by an associated processor, enable the respective device 800 to perform its intended tasks. Of course, the memory 820 may form a data buffer for data transmitted to and from the same. Exemplary embodiments of the system, subsystems, and modules as described herein may be implemented, at least in part, by computer software executable by the processor 810, or by hardware, or by combinations thereof.

The communication interface 830 modulates information for transmission by the respective apparatus 800 to another apparatus. The respective communication interface 830 is also configured to receive information from another processor for further processing. The communication interface 830 can support duplex operation for the respective other processor 810.

The system incorporates leading transplanters and haematologists, who want to exploit the data they have and use AI to tackle the big open questions for diseases such as AML for curative transplantation. They wanted to predict whether the NHS is under-referring AML patients for potentially curative transplants, or other remedial measures for other diseases.

The system takes into account all the uncertainties and variabilities that clinicians and patients are facing across the patient pathway—from diagnosis to the curative transplant. The model simulates, for instance, hundreds of thousands of possible AML patient journeys. This generates ranges for the numbers of patients per year who probably should receive a curative transplant. This was then compared to actual numbers of curative transplants over the last five years. The results showed the number of predicted eligible patients represents a 64-96% increase. That is an additional 202 to 305 patients every year.

With continuing reference to the description of the FIGUREs, a system (700) (and related method) has been introduced for predicting and prescribing a treatment (470) to a disease for a patient. The system (700) operable on a processor (810) and memory (820) for predicting and prescribing a treatment (470) to a disease for a patient, configured to collect population data (401) about the disease (via, for instance, a data collection module 710), build a population and patient model (400) based on the population data (401) (via, for instance, a population model builder 720), record patient factors (440) of the patient associated with the disease (via, for instance, a patient monitoring module 730), and anonymize the patient factors (440) by creating synthetic data of the patient factors (440) (via, for instance, a synthetic data module 740). The system (700) is also configured to merge the patient factors (440) into the population and patient model (400) (via, for instance, a patient model builder 750), prescribe the treatment (470) to the disease for the patient based on predicted outcomes from the population and patient model (400) (via, for instance, a treatment module 760), and store the treatment (470) to the disease for the patient in the memory (820) (via, for instance, a reporting module 770).

The population data (401) includes, without limitation, a diagnosed population (405), a population of patients under different treatments (410, 415), cured population (420), actual treatment population (425), and treatment eligible population (430). The patient factors (440) include, without limitation, patient diagnosis (445), patient treatment options (450, 455, 460), a cured patent (465), age of the patient (477), disease risk for the patient (479), fitness of the patient (481), and mortality rates (483). The patient factors (440) include clinical data including fitness of the patient (481) and fitness change of the patient (487) recorded from sensors connected to the patient (via, for instance, the patient monitoring module (730). The population data (401) includes an actual treatment population (425) and a treatment eligible population (430) and the system being configured to compare the actual treatment population (425) to the treatment eligible population (430) to determine a gap (435) therebetween to refine the population and patient model (400) (via, for instance, the population and patient model builder 720, 750).

As described above, the exemplary embodiments provide both a method and corresponding apparatus consisting of various modules providing functionality for performing the steps of the method. The modules may be implemented as hardware (embodied in one or more chips including an integrated circuit such as an application specific integrated circuit), or may be implemented as software or firmware for execution by a processor. In particular, in the case of firmware or software, the exemplary embodiments can be provided as a computer program product including a computer readable storage medium embodying computer program code (i.e., software or firmware) thereon for execution by the computer processor. The computer readable storage medium may be non-transitory (e.g., magnetic disks; optical disks; read only memory; flash memory devices; phase-change memory) or transitory (e.g., electrical, optical, acoustical or other forms of propagated signals-such as carrier waves, infrared signals, digital signals, etc.). The coupling of a processor and other components is typically through one or more busses or bridges (also termed bus controllers). The storage device and signals carrying digital traffic respectively represent one or more non-transitory or transitory computer readable storage medium. Thus, the storage device of a given electronic device typically stores code and/or data for execution on the set of one or more processors of that electronic device such as a controller.

Although the embodiments and its advantages have been described in detail, it should be understood that various changes, substitutions, and alterations can be made herein without departing from the spirit and scope thereof as defined by the appended claims. For example, many of the features and functions discussed above can be implemented in software, hardware, or firmware, or a combination thereof. Also, many of the features, functions, and steps of operating the same may be reordered, omitted, added, etc., and still fall within the broad scope of the various embodiments.

Moreover, the scope of the various embodiments is not intended to be limited to the embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized as well. Accordingly, the appended claims are intended to include within their scope such processes, machines, manufacture, compositions of matter, means, methods, or steps.

1. A system operable on a processor and memory for predicting and prescribing a treatment to a disease for a patient, configured to:
collect population data about said disease;
build a population and patient model based on said population data;
record patient factors of said patient associated with said disease;
anonymize said patient factors by creating synthetic data of said patient factors;
merge said patient factors into said population and patient model;
prescribe said treatment to said disease for said patient based on predicted outcomes from said population and patient model; and
store said treatment to said disease for said patient in said memory.

2. The system as recited in claim 1 wherein said population data (401) comprise a diagnosed population, a population of patients under different treatments, cured population, actual treatment population, and treatment eligible population.

3. The system as recited in claim 1 wherein said patient factors comprise patient diagnosis, patient treatment options, a cured patent, age of said patient, disease risk for said patient, fitness of said patient, and mortality rates.

4. The system as recited in claim 1 wherein said patient factors comprise fitness of said patient and fitness change of said patient recorded from sensors connected to said patient.

5. The system as recited in claim 1 wherein said population data comprises an actual treatment population and a treatment eligible population and the system being configured to compare said actual treatment population to said treatment eligible population to determine a gap therebetween to refine said population and patient model.

6. A method operable on a processor and memory for predicting and prescribing a treatment to a disease for a patient, comprising:
collecting population data about said disease;
building a population and patient model based on said population data;
recording patient factors of said patient associated with said disease;
anonymizing said patient factors by creating synthetic data of said patient factors;
merge said patient factors into said population and patient model;
prescribing said treatment to said disease for said patient based on predicted outcomes from said population and patient model; and
storing said treatment to said disease for said patient in said memory.

7. The method as recited in claim 6 wherein said population data (401) comprise a diagnosed population, a population of patients under different treatments, cured population, actual treatment population, and treatment eligible population.

8. The method as recited in claim 6 wherein said patient factors comprise patient diagnosis, patient treatment options, a cured patent, age of said patient, disease risk for said patient, fitness of said patient, and mortality rates.

9. The method as recited in claim 6 wherein said patient factors comprise fitness of said patient and fitness change of said patient recorded from sensors connected to said patient.

10. The method as recited in claim 6 wherein said population data comprises an actual treatment population and a treatment eligible population and the method comprising comparing said actual treatment population to said treatment eligible population to determine a gap therebetween to refine said population and patient model.

11. A system operable on a processor and memory for predicting and prescribing a curative transplant to Acute Myeloid Leukemia for a patient, configured to:
collect population data about said Acute Myeloid Leukemia;
build a population and patient model based on said population data;
record patent factors of said patient associated with said Acute Myeloid Leukemia;
anonymize said patient factors by creating synthetic data of said patent factors;
merge said patent factors into said population and patient model;
prescribe said curative transplant to said Acute Myeloid Leukemia for said patient based on predicted outcomes from said population and patient model; and
store said curative transplant to said Acute Myeloid Leukemia for said patient in said memory.

12. The system as recited in claim 11 wherein said population data comprises diagnosed population, palliative care population, chemotherapy population, remission population, actual curative transplant population, and curative transport eligible population.

13. The system as recited in claim 11 wherein said patient factors comprise patient diagnosis, patient palliative care option, patient intensive chemotherapy option, patient non-intensive chemotherapy option, patient remission, age of said patient, disease risk for said patient, fitness of said patient, and mortality rates.

14. The system as recited in claim 11 wherein said patient factors comprise fitness of said patient and fitness change of said patient recorded from sensors connected to said patient.

15. The system as recited in claim 11 wherein said population data comprises actual curative transplant population and curative transport eligible population and the system is configured to compare said actual curative transplant population to said curative transport eligible population to determine a gap therebetween to refine said population and patient model.

16. A method operable on a processor and memory for predicting and prescribing a curative transplant to Acute Myeloid Leukemia for a patient, comprising:
collecting population data about said Acute Myeloid Leukemia;
building a population and patient model based on said population data;
recording patent factors of said patient associated with said Acute Myeloid Leukemia;
anonymizing said patient factors by creating synthetic data of said patent factors;
merging said patent factors into said population and patient model;
prescribing said curative transplant to said Acute Myeloid Leukemia for said patient based on predicted outcomes from said population and patient model; and
storing said curative transplant to said Acute Myeloid Leukemia for said patient in said memory.

17. The method as recited in claim 16 wherein said population data comprises diagnosed population, palliative care population, chemotherapy population, remission population, actual curative transplant population, and curative transport eligible population.

18. The method as recited in claim 16 wherein said patient factors comprise patient diagnosis, patient palliative care option, patient intensive chemotherapy option, patient non-intensive chemotherapy option, patient remission, age of said patient, disease risk for said patient, fitness of said patient, and mortality rates.

19. The method as recited in claim 16 wherein said patient factors comprise fitness of said patient and fitness change of said patient recorded from sensors connected to said patient.

20. The method as recited in claim 16 wherein said population data comprises actual curative transplant population and curative transport eligible population and the method comprising comparing said actual curative transplant population to said curative transport eligible population to determine a gap therebetween to refine said population and patient model.
